(19) United States
(12) Patent Application Publication
(10) Pub. No.: US 2007/0105113 A1
(43) Pub. Date: May 10, 2007

(54) Methods of degrading dsrna and synthesizing rna

(75) Inventors: Hiroaki Sagawa, Shiga (JP); Jun Tomono, Okayama (JP); Harumi Ueno, Shiga (JP); Ikunoshin Kato, Shiga (JP)

Correspondence Address: BROWDY AND NEIMARK, P.L.L.C.;624 NINTH STREET, NW, SUITE 300, WASHINGTON, DC, 20001-5303, US

(73) Assignee: TAKARA BIO INC., Otsu-shi (JP)

(21) Appl. No.: 10/567,731

(22) PCT Filed: Aug. 10, 2004
(86) PCT No.: PCT/JP04/11480

§ 371 Date: Feb. 10, 2006

(30) Foreign Application Priority Data
Aug. 14, 2003 (JP) 2003-293553
Sep. 30, 2003 (JP) 2003-342126
Dec. 8, 2003 (JP) 2003-409639
Mar. 24, 2004 (JP) 2004-086129

(51) Int. Cl. C12Q 1/68; C12P 19/34; C12N 9/22

(52) U.S. Cl. 435/6; 435/91.2; 435/199

(57) ABSTRACT

A protein having an activity of degrading a dsRNA, namely, being capable of acting on a long-chain dsRNA to form a dsRNA of a definite length; a method of efficiently preparing a dsRNA of a definite length which comprises treating a dsRNA with the protein having an activity of degrading a dsRNA in the coexistence of a protein having an activity of binding to a nucleic acid such as a protein having an RNA-binding activity; and a method of using the protein having an activity of binding to a nucleic acid to elevate the efficiency in an RNA synthesis reaction typified by dsRNA synthesis.

### TECHNICAL FIELD

The present invention relates to a protein having an activity of degrading a dsRNA which has an activity of generating a dsRNA of a specific length, a method for efficiently degrading a dsRNA using said protein and a protein having an activity of binding to a nucleic acid (e.g., a protein having an activity of binding to an RNA) in combination, as well as a method for efficiently synthesizing an RNA using a protein having an activity of binding to a nucleic acid and a protein having an activity of synthesizing an RNA in combination.

### BACKGROUND ART

Recently, genetic engineering techniques that utilize small dsRNAs have been reported.

For example, RNA interference (RNAi) is a phenomenon in which an mRNA is degraded with a dsRNA in a sequence-specific manner, resulting in suppression of gene expression. The beginning of the finding that a dsRNA can be used for gene silencing was a study in which an antisense was used in Caenorhabditis elegans. In 1995, Guo and Kemphues carried out an experiment to suppress a gene called par-1 using an antisense RNA. When an antisense RNA was added, the expression of par-1 was suppressed as expected. Surprisingly, a sense RNA which was used as a control also suppressed the expression of par-1, resulting in a phenotype of a par-1 mutant (see, for example, Non-patent Document 1).

This contradiction was resolved by Fire et al. in 1998. When an antisense RNA or a sense RNA is synthesized using an RNA polymerase, a small amount of an inverse RNA is unintentionally produced in a nonspecific manner. It has been shown that a dsRNA formed due to the contamination is the essential entity for the gene silencing, an antisense RNA or a sense RNA cannot suppress gene expression, and a dsRNA formed by annealing an antisense RNA to a sense RNA can efficiently suppress gene expression (e.g., Non-patent Document 2).

In RNA interference, an enzyme called a Dicer produces a small RNA (a short interfering RNA (siRNA)) from a dsRNA (e.g., Non-patent Document 3).

It is considered that the siRNA produced as a result of the action of the enzyme is incorporated into a complex called an RNA induced silencing complex (RISC), and the complex recognizes and degrades a target mRNA. However, the exact functions of the respective factors supposed to be involved in RNA interference have been unknown in many cases (e.g., Non-patent Document 4).

It is importance to efficiently produce a dsRNA or an siRNA in order to efficiently carry out RNA interference. The Dicer is exemplified by human Dicer (e.g., Non-patent Document 5). A recombinant Dicer (e.g., Non-patent Document 6) is sold by Gene Therapy Systems or Stratagene.

However, it has not been examined in detail whether or not the inherent enzymatic properties of the above-mentioned recombinant Dicer are sufficiently exhibited. Furthermore, the minimal domain of the Dicer to be included for retaining its activity of producing a preferable dsRNA (siRNA) upon its action on a dsRNA, or for increasing the productivity thereof using genetic engineering techniques is not known well.

In addition, a particularly effective method for efficient production of a dsRNA has not been known.

A method in which a dsRNA of about 21 nucleotides in length synthesized using an RNA polymerase is utilized as it is as an siRNA has been reported (e.g., Non-patent Document 7). A method by which an RNA can be efficiently produced can be utilized for the above-mentioned method, if it is available.

Non-patent Document 1: Guo, S. et al., Cell, 1995, vol. 81, p. 611-620

Non-patent Document 2: Fire, A. et al., Nature, 1998, vol. 39, p. 806-811

Non-patent Document 3: Bernstein, E. et al., Nature, 2001, vol. 409, p. 363-366

Non-patent Document 4: Tabara, H. et al., Cell, 2002, vol. 109, p. 861-871

Non-patent Document 5: Zhang, H. et al., The EMBO Journal, 2002, vol. 21, No. 21, p. 5875-5885

Non-patent Document 6: Myers, J. W. et al., Nature Biotechnology, 2003, vol. 21, p. 324-328

Non-patent Document 7: Donze, O. et al., Nucleic Acids Research, 2002, vol. 30, No. 10, e46

### DISCLOSURE OF INVENTION

Problems to be Solved by the Invention

The problems to be solved by the present invention are to provide a protein having an activity of degrading a dsRNA which has an activity of producing a dsRNA of a specific length, as well as to provide a method for efficiently producing a dsRNA of a specific length which can be utilized for RNA interference or the like, and a method for promoting RNA synthesis.

Means to Solve the Problems

The present inventors have studied intensively in order to solve the above-mentioned problems. As a result, the present inventors have found, by analyzing a functional domain of a Dicer, a protein having an activity of degrading a dsRNA which has an activity of acting on a long dsRNA to produce a dsRNA of a specific length. Furthermore, the present inventors have found that a dsRNA of a specific length can be efficiently prepared by allowing a protein having an activity of degrading a dsRNA to act on a dsRNA in the presence of a protein having an activity of binding to a nucleic acid (e.g., a protein having an activity of binding to an RNA), and that the protein having an activity of binding to a nucleic acid also increases the efficiency of a reaction of RNA synthesis, the representative of which is dsRNA synthesis. Thus, the present invention has been completed.

The first aspect of the present invention relates to a protein having an activity of degrading a dsRNA, which has an activity of acting on a dsRNA to produce a dsRNA of a specific length.

According to the first aspect, the protein having an activity of degrading a dsRNA preferably has a functional domain of a Dicer. For example, it preferably consists of RNase IIIa, RNase IIIb and dsRNA-binding domains. The protein may further contain a PAZ domain. It is possible to produce a dsRNA of about 15 to 30 base pairs as the dsRNA of a specific length by using the protein of the first aspect. The protein having an activity of degrading a dsRNA of the first aspect is exemplified by a protein consisting of the amino acid sequence of SEQ ID NO:4 or 17, or an amino acid sequence encoded by the nucleotide sequence of SEQ ID NO:3 or 16. Alternatively, the protein may be a protein consisting of an amino acid sequence in which one or plural amino acid(s) is(are) substituted, deleted, inserted or added in the amino acid sequence of SEQ ID NO:4 or 17. The protein having an activity of degrading a dsRNA of the first aspect can be efficiently produced by converting a codon into one that is suitable for expression in a host, or by reinforcing a host to be used for a rare codon.

Furthermore, the protein can be expressed using a cold-inducible vector. In addition, the protein of the first aspect may be contained in a kit as a component.

The second aspect of the present invention relates to a method for degrading a dsRNA, the method comprising allowing a protein having an activity of degrading a dsRNA to act on a dsRNA in the presence of a protein having an activity of binding to a nucleic acid to produce a dsRNA of a specific length.

According to the second aspect, the protein having an activity of binding to a nucleic acid and the protein having an activity of degrading a dsRNA may be a fusion protein. The protein having an activity of binding to a nucleic acid may be a protein having an activity of binding to an RNA. The protein having an activity of binding to an RNA may be a cold shock protein. The cold shock protein may be derived from a thermophilic bacterium or a thermostable bacterium. Although it is not intended to limit the present invention, for example, the cold shock protein is exemplified by cold shock protein B from Thermotoga maritima.

According to the method of the second aspect, it is possible to produce a dsRNA of about 15 to 30 base pairs as the dsRNA of specific length. Furthermore, according to the second aspect, the protein having an activity of degrading a dsRNA may be the protein of the first aspect, or a native Dicer or a functional equivalent thereof.

The third aspect of the present invention relates to a method for synthesizing an RNA, the method comprising conducting an RNA synthesis reaction using a protein having an activity of synthesizing an RNA in the presence of a protein having an activity of binding to a nucleic acid.

According to the third aspect, the protein having an activity of binding to a nucleic acid and the protein having an activity of synthesizing an RNA may be a fusion protein. The protein having an activity of binding to a nucleic acid may be a cold shock protein. The cold shock protein may be derived from a thermophilic bacterium or a thermostable bacterium. Although it is not intended to limit the present invention, for example, the cold shock protein is exemplified by cold shock protein B from Thermotoga maritima. The protein having an activity of synthesizing an RNA may be a DNA-dependent RNA polymerase.

The fourth aspect of the present invention relates to a composition used for the method of the second aspect, the composition containing a protein having an activity of binding to a nucleic acid and a protein having an activity of degrading a dsRNA.

The fifth aspect of the present invention relates to a kit used for the method of the second aspect, the kit containing a protein having an activity of binding to a nucleic acid and a protein having an activity of degrading a dsRNA.

The sixth aspect of the present invention relates to a composition used for the method of the third aspect, the composition containing a protein having an activity of binding to a nucleic acid and a protein having an activity of synthesizing an RNA.

The seventh aspect of the present invention relates to a kit used for the method of the third aspect, the kit containing a protein having an activity of binding to a nucleic acid and a protein having an activity of synthesizing an RNA.

### EFFECTS OF THE INVENTION

The present invention provides a protein having an activity of degrading a dsRNA which can be used to prepare a dsRNA of a specific length. Furthermore, a dsRNA of a specific length which can be utilized for RNA interference or the like can be efficiently produced according to the present invention.

### BEST MODE FOR CARRYING OUT THE INVENTION

As used herein, a Dicer refers to a protein that has a function by which a long dsRNA can be processed into an siRNA at an early stage of RNAi. Examples of native Dicers include, but are not limited to, one composed of, from the N terminus, an ATP-binding domain, an RNA helicase domain, a PAZ domain of unknown function, RNase IIIa and RNase IIIb domains, and a dsRNA-binding domain.

As used herein, a functional domain of a Dicer refers to a part that encodes a region involved in an activity by which a dsRNA of a specific length can be produced upon action on a long dsRNA.

Examples of the functional domains include, but are not limited to, one consisting of RNase IIIa, RNase IIIb and dsRNA-binding domains. The RNase IIIa and RNase IIIb domains may be a part that encodes a region involved in an activity of specifically acting on a double-stranded RNA to produce an oligonucleotide of a specific length having a phosphate group at the 5′ end as described in Zhang, H. et al., The EMBO Journal, 2002, vol. 21, No. 21, p. 5875-5885. The dsRNA-binding domain may be a part that encodes an activity of specifically binding to a double-stranded RNA.

As used herein, a dsRNA refers to an RNA forming a double-stranded structure between an mRNA to be subjected to RNA interference and an RNA having a nucleotide sequence complementary to the mRNA.

The main exemplary application of a product of a dsRNA degradation reaction according to the present invention is an siRNA as described below.

As used herein, a dsRNA of a specific length refers to a dsRNA of a specific length, for example, from about 10 to 100 base pairs although it is not intended to limit the present invention. It may be a dsRNA of a specific length from about 15 to 30 base pairs, particularly from 20 to 25 base pairs. Such a dsRNA can be used as an siRNA.

As used herein, a protein having an activity of binding to a nucleic acid refers to a protein having an activity of binding to a single-stranded or double-stranded DNA or RNA. The protein preferably has a function of canceling a secondary structure of a nucleic acid. Examples thereof include DNA helicases, RNA helicases and functional equivalents thereof.

As used herein, a cold-inducible vector refers to a vector having a promoter that is capable of functioning at a low temperature. Examples thereof include the pCold-series vectors as described in WO 99/27117.

As used herein, cold shock proteins generically refer to proteins expressed as a result of stimulation by lowered temperature as compared with the inherent growth conditions.

As used herein, complete degradation of a long dsRNA as a substrate refers to degradation to a degree that the uncleaved long dsRNA as a substrate is not observed after a degradation reaction as determined by electrophoresis.

As used herein, a PAZ domain is a domain between an RNA helicase domain and RNase IIIa and RNase IIIb domains in a Dicer. For example, it is a region from amino acid 898 to amino acid 1064 in the amino acid sequence of human Dicer of SEQ ID NO:1 (from nucleotide 2692 to nucleotide 3192 in SEQ ID NO:2).

According to the present invention, a rare codon means a codon whose usage is low. A single amino acid may be prescribed by plural codons. The usages of the plural codons may be biased depending on the biological species. It is known that the amount of a transfer RNA (tRNA) corresponding to a codon of low usage is generally small, and expression efficiency is decreased if an amino acid sequence encoded by a nucleotide sequence that contains a rare codon is to be expressed. Thus, a protein can be efficiently produced by reinforcement for a rare codon (e.g., by increasing the amount of a tRNA corresponding to a rare codon).

Hereinafter, the present invention will be described in detail.

(1) Protein Having an Activity of Degrading a dsRNA of the Present Invention, Method for Producing the Protein, and Kit Containing the Protein

The protein having an activity of degrading a dsRNA of the present invention can act on a dsRNA to produce a dsRNA of a specific length. There is no specific limitation concerning the protein having an activity of degrading a dsRNA as long as it can produce a dsRNA of a specific length from a long dsRNA. It is exemplified by a protein having a functional domain of a Dicer. The functional domain of a Dicer may be a protein consisting of RNase IIIa, RNase IIIb and dsRNA-binding domains. Any protein may be used regardless of its origin as long as it can produce a dsRNA of a specific length from a long dsRNA.

There is no specific limitation concerning the RNase IIIa, RNase IIIb and dsRNA-binding domains. In case of human Dicer, an exemplary one has amino acid 1271 to amino acid 1924 in the N-terminal portion of the amino acid sequence of SEQ ID NO:1 (nucleotide 3811 to nucleotide 5772 in the nucleotide sequence of SEQ ID NO:2). Examples thereof include a protein (a Dicer mutant) consisting of the amino acid sequence of SEQ ID NO:4 or an amino acid sequence encoded by the nucleotide sequence of SEQ ID NO:3, and a protein (a Dicer mutant) consisting of the amino acid sequence of SEQ ID NO:12 or an amino acid sequence encoded by the nucleotide sequence of SEQ ID NO:13.

Since the Dicer is a large protein, it is not suitable for production of a recombinant. On the other hand, the protein having an activity of degrading a dsRNA of the present invention is smaller than the native enzyme and compact. Thus, it is useful for production of a recombinant. In general, if an enzyme derived from a higher organism (e.g., human) is to be produced as a recombinant in cells of a bacterium (e.g., Escherichia coli), it may be difficult to produce the recombinant while retaining an equivalent enzymatic activity in many cases. Accordingly, the protein having an activity of degrading a dsRNA of the present invention is very useful for production in cells from an organism other than its origin.

The protein of the present invention may contain a PAZ domain. Examples thereof include, but are not limited to, a protein having amino acid 898 to amino acid 1924 in the amino acid sequence of SEQ ID NO:1 (nucleotide 2692 to nucleotide 5772 in SEQ ID NO:2), a protein (a Dicer mutant) consisting of the amino acid sequence of SEQ ID NO:17 or an amino acid sequence encoded by the nucleotide sequence of SEQ ID NO:16, and a protein (a Dicer mutant) consisting of the amino acid sequence of SEQ ID NO:18 or an amino acid sequence encoded by the nucleotide sequence of SEQ ID NO:19.

Furthermore, a more enzymatically stable one can be obtained as a mutant. Although it is not intended to limit the present invention, for example, an increased stability may be observed in case of the PAZ domain+the RNase III domain as compared with a mutant protein composed only of the RNase III domain. The activity can be retained even after more freeze-thaw cycles, and the activity can be retained in liquid state for a longer period of time using a certain storage buffer.

Although it is not intended to limit the present invention, for example, the protein of the present invention can degrade a long dsRNA to produce an siRNA that is effective for RNA interference.

The proteins having an activity of degrading a dsRNA of the present invention include one in which one or plural amino acid(s) (or nucleotide(s)) is(are) substituted, deleted, inserted or added in the amino acid sequence (or the nucleotide sequence) as long as it has the above-mentioned function.

Although it is not intended to limit the present invention, the proteins having an activity of degrading a dsRNA of the present invention include ones having the following being added to the protein: a sequence derived from an expression vector such as an expression or translation enhancing sequence (e.g., Perfect DB sequence), or an amino acid sequence such as a tag sequence for purification of an expressed protein (e.g., His tag sequence) or a sequence for removing an N-terminal additional sequence of an expressed protein (e.g., Factor Xa sequence). Examples of such proteins include, but are not limited to, a protein having an activity of degrading a dsRNA that has the amino acid sequence of SEQ ID NO:12 or 18.

The protein having an activity of degrading a dsRNA of the present invention can convert a long dsRNA into a dsRNA of a specific length. Thus, one can prepare a dsRNA of a desired length according to the present invention by appropriately selecting the protein having an activity of degrading a dsRNA to be used. Although it is not intended to limit the present invention, the dsRNA of a specific length is exemplified by a dsRNA of a specific length from about 10 to 100 base pairs, preferably from about 15 to 30 base pairs, more preferably from about 20 to 25 base pairs.

A dsRNA used as a substrate is not completely degraded using a conventional commercially available enzyme. On the other hand, a long dsRNA as a substrate can be substantially wholly cleaved using the protein having an activity of degrading a dsRNA of the present invention. Since the dsRNA as a substrate can be wholly cleaved and the RNA interference activity of the degradation product is equivalent, scaling down of the long dsRNA as a substrate and, in its turn, omission of a step of removing undegraded dsRNA are made possible.

Furthermore, the activity of the protein having an activity of degrading a dsRNA of the present invention (for example, without limitation, a Dicer mutant) can be stably retained for a long period of time by storage in a tris-hydrochloride buffer at pH 8.5 or above in the presence of magnesium chloride either at 4° C. or at −20° C.

The protein having an activity of degrading a dsRNA of the present invention may be in a form of a fusion protein with a protein having an activity of binding to a nucleic acid as described in (2) below (for example, without limitation, a protein having an activity of binding to an RNA). Although it is not intended to limit the present invention, examples thereof include a fusion protein of the above-mentioned Dicer mutant and a protein having an activity of binding to a nucleic acid (e.g., a protein having an activity of binding to an RNA).

A method for producing the protein having an activity of degrading a dsRNA of the present invention may comprise conversion of a codon into one that is suitable for expression in a host, or reinforcement for a rare codon. Although it is not intended to limit the present invention, for example, it may be a production method comprising modification of a codon in a gene to be expressed. Expression can be carried out using a gene in a state in which codons for an amino acid sequence of a protein are partially or entirely converted into ones optimal for protein expression, or using a host in a state in conformity therewith. Although it is not intended to limit the present invention, the host in a state in conformity with the state of optimal codons is exemplified by a host in which the amount of a tRNA that recognizes a specific codon is made several times larger than the amount normally produced in the cell using genetic engineering techniques. Examples of such hosts include Escherichia coli cells such as an Escherichia coli cell reinforced with tRNAs for codons for arginine (AGA, AGG), and an Escherichia coli cell reinforced with tRNAs for isoleucine (AUA), proline (CCC) and leucine (CUA).

A method in which expression of a protein of interest in a host is increased by modification of a codon may be used. In this case, one may take the secondary structure of an mRNA into consideration.

There is no specific limitation concerning the method as long as protein expression is increased by conversion of a codon in a gene to be expressed, reinforcement or the like according to the method.

There is no specific limitation concerning a vector used for producing the protein having an activity of degrading a dsRNA of the present invention. Any commercially available vector or expression system may be used. In particular, the pET system (Novagen) can be used, for example, although it is not intended to limit the present invention. In addition, a vector having a promoter that is capable of functioning at a low temperature can be preferably used. Examples thereof include the pCold-series vectors as described in WO 99/27117.

Any vector can be preferably used according to the production method of the present invention as long as the vector can be used to express a protein retaining a function by which a dsRNA of a specific length can be produced.

Furthermore, a vector that is capable of expressing a protein that is in a form of inclusion body upon expression of the protein but whose function can be restored by a subsequent refolding procedure may be included, provided that it is possible to obtain a protein that can finally retain a function by which a dsRNA of a specific length can be produced.

In an embodiment of the method of the present invention, for example, where the above-mentioned Dicer mutant is to be produced, the productivity and the yield of the protein in an activity-retaining form can be increased as compared with a case where the full-length conventional human Dicer is expressed.

The methods for producing the protein having an activity of degrading a dsRNA of the present invention include a method in which the protein is expressed in a form of a fusion protein with a protein having an activity of binding to a nucleic acid (for example, without limitation, a protein having an activity of binding to an RNA) as described in (2) below.

(2) Method for Promoting dsRNA Degradation Using the Protein Having an Activity of Binding to a Nucleic Acid Of the Present Invention, and Method for Promoting RNA Synthesis Using the Protein

The method for promoting dsRNA degradation of the present invention in which a dsRNA of a specific length is produced is characterized in that it is carried out in the presence of a protein having an activity of binding to a nucleic acid. There is no specific limitation concerning the protein having an activity of binding to a nucleic acid as long as it results in promotion of a dsRNA degradation activity. For example, the above-mentioned protein having an activity of binding to an RNA can be preferably used.

Examples of the proteins having an activity of binding to an RNA include, but are not limited to, a cold shock protein (Csp). In particular, a cold shock protein that can function in a normal temperature range can be preferably used. A cold shock protein derived from a thermophilic bacterium or a thermostable bacterium is preferable. Although it is not intended to limit the present invention, for example, a CspB protein derived from Thermotoga maritima having the amino acid sequence of SEQ ID NO:9 (an amino acid sequence encoded by the nucleotide sequence of SEQ ID NO:10) can be preferably used. For example, a recombinant of the CspB protein from Thermotoga maritima can be produced using genetic engineering techniques according to the method described in Protein Science, 8:394-403 (1999) with Thermotoga maritima strain MSB8 purchased from Deutsche Sammlung von Mikroorganismen und Zellkulturen GmbH (DSM3109). A vector having a promoter capable of functioning at a low temperature can be preferably used. Examples thereof include the pCold-series vectors as described in WO 99/27117.

It is possible to promoter an activity of degrading a dsRNA by using a CspB protein and a protein having an activity of degrading a dsRNA in combination. According to the method of the present invention, it is possible to promote an activity of producing a dsRNA of a specific length for any of the proteins having an activity of degrading a dsRNA to produce a dsRNA of a specific length as described in (1) above (e.g., functional equivalents such as Dicer mutants, native Dicers or commercially available recombinant Dicers).

An activity of degrading a dsRNA can be promoted according to the method of the present invention by using a protein having an activity of binding to a nucleic acid and a protein having an activity of degrading a dsRNA in combination. The resulting degradation product has an RNA interference activity (per unit weight) equivalent to that of a degradation product obtained without using a protein having an activity of binding to a nucleic acid in combination. Thus, the use of a protein having an activity of binding to a nucleic acid is very useful for RNA interference.

A dsRNA used as a substrate is not completely degraded according to a conventional method. On the other hand, a long dsRNA as a substrate can be substantially wholly cleaved according to the method of the present invention. Since the dsRNA as a substrate can be wholly cleaved and the RNA interference activity of the degradation product is equivalent, scaling down of the long dsRNA as a substrate and, in its turn, omission of a step of removing undegraded dsRNA are made possible.

Furthermore, according to the method of the present invention, a protein having an activity of binding to a nucleic acid (e.g., a protein having an activity of synthesizing an RNA) may be in a form of a fusion protein with a protein having an activity of degrading a dsRNA.

The method for promoting RNA synthesis of the present invention is characterized in that it is carried out in the presence of a protein having an activity of binding to a nucleic acid. There is no specific limitation concerning the protein having an activity of binding to a nucleic acid as long as it results in promotion of an RNA synthesis activity of a protein having the activity. A protein having an activity of binding to a nucleic acid, a cold shock protein (Csp), can be preferably used. Although it is not intended to limit the present invention, a cold shock protein derived from a thermophilic bacterium or a thermostable bacterium can be preferably used. Examples of the cold shock proteins include, but are not limited to, a CspB protein derived from Thermotoga maritima having the amino acid sequence of SEQ ID NO:9 (the nucleotide sequence of SEQ ID NO:10).

For example, an RNA synthesis activity of an RNA polymerase can be promoted by including a CspB protein in an RNA synthesis system. The protein can promote the synthesis activity whether the product is a single-stranded RNA or a double-stranded RNA.

The method for promoting RNA synthesis of the present invention can be utilized for synthesis of not only a long dsRNA but also a short dsRNA (e.g., an siRNA). The method can be utilized for synthesizing, using T7 RNA polymerase or the like, an siRNA preferably of about 15 to 30 base pairs, more preferably of about 20 to 25 base pairs although it is not intended to limit the present invention.

The protein having an activity of binding to a nucleic acid promotes two types of reactions, i.e., dsRNA synthesis with a protein having an RNA synthesis activity and dsRNA degradation with a protein having a dsRNA degradation activity, according to the method of the present invention. Thus, the method can be utilized for a system for synthesizing a dsRNA which is particularly important for RNA interference or a system for producing an siRNA. In this case, the respective proteins may be individual ones or they may be in a form of a fusion protein.

(3) Composition Used for the Method of the Present Invention

The composition of the present invention is a composition for efficiently conducting a reaction of degradation into a dsRNA of a specific length and/or an RNA synthesis reaction.

The composition contains a protein having an activity of binding to a nucleic acid as described in (2) above. A cold shock protein can be preferably used as the protein having an activity of binding to a nucleic acid although it is not intended to limit the present invention. For example, a cold shock protein (Csp) derived from a thermophilic bacterium or a thermostable bacterium can be preferably used. A CspB protein derived from Thermotoga maritima having the amino acid sequence of SEQ ID NO:9 (an amino acid sequence encoded by the nucleotide sequence of SEQ ID NO:10) is preferable.

The composition of the present invention may contain a protein having an activity of degrading a dsRNA and/or a protein having an activity of synthesizing an RNA. One having a functional domain of a Dicer is preferable as a protein having an activity of degrading a dsRNA that is capable of producing a dsRNA of a specific length. For example, a protein consisting of RNase IIIa, RNase IIIb and dsRNA-binding domains can be preferably used. Although it is not intended to limit the present invention, for example, any of the functional equivalents such as Dicer mutants, native Dicers or commercially available recombinant Dicers as described in (1) above may be used. The composition containing a Dicer mutant may be a composition containing a protein consisting of the amino acid sequence of SEQ ID NO:4 or 17 or an amino acid sequence encoded by the nucleotide sequence of SEQ ID NO:3 or 16. It may be a composition containing a protein consisting of an amino acid sequence in which one or plural amino acid(s) is(are) substituted, deleted, inserted or added in the amino acid sequence of SEQ ID NO:4 or 17.

The protein having an activity of degrading a dsRNA may be a protein having the following being added to the protein: a sequence derived from an expression vector such as an expression or translation enhancing sequence (e.g., Perfect DB sequence), or an amino acid sequence such as a tag sequence for purification of an expressed protein (e.g., His tag sequence) or a sequence for removing an N-terminal additional sequence of an expressed protein (e.g., Factor Xa sequence). Examples of such proteins include, but are not limited to, a protein having an activity of degrading a dsRNA that has the amino acid sequence of SEQ ID NO:12 or 18. Furthermore, the composition of the present invention may contain a buffer for stabilizing a Dicer mutant as described in (1) above.

For example, T7 RNA polymerase, T3 RNA polymerase, SP6 RNA polymerase or the like can be preferably used as a protein having an activity of synthesizing an RNA.

In another embodiment of the composition of the present invention, the composition may contain a fusion protein of a protein having an activity of binding to a nucleic acid as described in (2) above (e.g., a protein having an activity of binding to an RNA) and a protein having an activity of degrading a dsRNA that is capable of producing a dsRNA of a specific length and/or a fusion protein of the protein having an activity of binding to a nucleic acid and a protein having an activity of synthesizing an RNA.

A reaction of efficient degradation into a dsRNA of a specific length and/or synthesis of a single-stranded or double-stranded RNA can be conveniently carried out using the composition of the present invention.

An embodiment of the composition of the present invention is a composition that can be utilized for synthesis of not only a long dsRNA but also a short dsRNA (e.g., an siRNA). Such a composition is effective for synthesizing a dsRNA of about 10 to 100 base pairs, preferably of about 15 to 30 base pairs, more preferably of about 20 to 25 base pairs.

(4) Kit Used for the Method of the Present Invention

The kit used for the method of the present invention is a kit for efficiently conducting a reaction of degradation into a dsRNA of a specific length and/or an RNA synthesis reaction.

The kit contains a protein having an activity of binding to a nucleic acid as described in (2) above. A cold shock protein can be preferably used as the protein having an activity of binding to a nucleic acid although it is not intended to limit the present invention. For example, a cold shock protein, (Csp) derived from a thermophilic bacterium or a thermostable bacterium can be preferably used. A CspB protein derived from Thermotoga maritima having the amino acid sequence of SEQ ID NO:9 can be preferably used.

The kit of the present invention may contain a protein having an activity of degrading a dsRNA which as an activity of producing a dsRNA of a specific length and/or a protein having an activity of synthesizing an RNA. One as described in (3) above can be preferably used as the protein having an activity of degrading a dsRNA and/or the protein having an activity of synthesizing an RNA. Furthermore, the kit of the present invention may contain a buffer for stabilizing a Dicer mutant as described in (1) above.

In another embodiment of the kit of the present invention, the kit may contain a fusion protein of a protein having an activity of binding to a nucleic acid as described in (2) above (e.g., a protein having an activity of binding to an RNA) and a protein having an activity of degrading a dsRNA that is capable of producing a dsRNA of a specific length and/or a fusion protein of the protein having an activity of binding to a nucleic acid and a protein having an activity of synthesizing an RNA.

The kit of the present invention may contain a component other than those as described above such as a reagent for purifying a dsRNA of a specific length produced as a result of the reaction, or a reagent for transferring it into a biological sample. Although it is not intended to limit the present invention, for example, it may contain a reagent for purifying an siRNA of about 21 base pairs, a regent for transferring it into a biological sample or the like.

A reaction of efficient degradation into a dsRNA of a specific length and/or synthesis of an RNA can be conveniently carried out using the kit of the present invention.

An embodiment of the kit of the present invention is a kit that can be utilized for synthesis of not only a long dsRNA but also a short dsRNA (e.g., an siRNA). Such a kit is effective for synthesizing a dsRNA of about 10 to 100 base pairs, preferably of about 15 to 30 base pairs, more preferably of about 20 to 25 base pairs.

### EXAMPLES

The following Examples illustrate the present invention in more detail, but are not to be construed to limit the scope thereof.

Among the procedures described herein, basic procedures including preparation of plasmids and restriction enzyme digestion were carried out as described in T. Maniatis et al. (eds.), Molecular Cloning: A Laboratory Manual 3rd ed., Cold Spring Harbor Laboratory (2001).

### Example 1

### Expression of RNase III Domain of Human Dicer

(1) Construction of Expression Vector

An expression vector was constructed as follows in order to express a polypeptide consisting of amino acid 1271 to amino acid 1924 (nucleotide 3811 to nucleotide 5772) in the N-terminal portion of the amino acid sequence of human Dicer as shown in SEQ ID NO:1.

First, synthetic primers 1 and 2 (SEQ ID NOS:5 and 6) were synthesized using a DNA synthesizer based on the nucleotide sequence available to the public under GenBank accession no. AB028449, and purified according to a conventional method. The synthetic primer 1 is a synthetic DNA that has a recognition sequence for a restriction enzyme KpnI at nucleotide 9 to nucleotide 14, and a nucleotide sequence corresponding to amino acid 1271 to amino acid 1277 in the amino acid sequence of human Dicer (SEQ ID NO:1) at nucleotide 16 to nucleotide 36. The synthetic primer 2 has a recognition sequence for a restriction enzyme HindIII at nucleotide 9 to nucleotide 14 and a nucleotide sequence corresponding to amino acid 1919 to amino acid 1924 in the amino acid sequence of human Dicer (SEQ ID NO:1) at nucleotide 18 to nucleotide 36.

A PCR was conducted using the synthetic primers. The reaction conditions for the PCR were as follows.

Briefly, a reaction mixture of a total volume of 50 μl was prepared by adding 2 μl of a template DNA (human cDNA library, human pancreas, Takara Bio), 5 μl of 10×LA PCR buffer (Takara Bio), 5 μl of dNTPs (Takara Bio), 10 pmol of the synthetic primer 1, 10 pmol of the synthetic primer 2, 0.5 U of Takara LA Taq (Takara Bio) and sterile water. The reaction mixture was placed in TaKaRa PCR Thermal Cycler SP (Takara Bio) and subjected to a reaction as follows: 30 cycles of 94° C. for 1 minute, 55° C. for 1 minute and 72° C. for 3 minutes.

After reaction, 5 μl of the reaction mixture was subjected to electrophoresis on 1.0% agarose gel. The observed about 2-kbp DNA fragment of interest was recovered and purified from the electrophoresis gel and subjected to ethanol precipitation. After ethanol precipitation, the recovered DNA was suspended in 5 μl of sterile water, and doubly digested with restriction enzymes KpnI (Takara Bio) and HindIII (Takara Bio). The KpnI-HindIII digest was extracted and purified after electrophoresis on 1.0% agarose gel to obtain a KpnI-HindIII-digested DNA fragment.

Next, a vector pCold08NC2 was prepared according to the method described in Examples 1-6 of WO 99/27117.

The vector pCold08 was cleaved with the same restriction enzymes as those used upon preparation of the KpnI-HindIII-digested DNA fragment and the termini were dephosphorylated. The thus prepared vector and the KpnI-HindIII-digested DNA fragment were mixed together and ligated to each other using DNA ligation kit (Takara Bio). 20 μl of the ligation mixture was used to transform Escherichia coli JM109. Transformants were grown on LB medium (containing 50 μg/ml of ampicillin) containing agar at a concentration of 1.5% (w/v).

A plasmid having the inserted DNA fragment of interest was confirmed by sequencing. This recombinant plasmid was designated as pCold08 hDi-R. This plasmid is designated and indicated as pCold08 hDi-R and has been deposited under accession no. FERM BP-10074 at International Patent Organism Depositary, National Institute of Advanced Industrial Science and Technology, AIST Tsukuba Central 6, 1-1, Higashi 1-chome, Tsukuba-shi, Ibaraki 305-8566, Japan since Aug. 11, 2003 (date of original deposit). pCold08 hDi-R is a plasmid containing a nucleotide sequence that encodes an amino acid sequence from amino acid 1271 to amino acid 1924 in the amino acid sequence of human Dicer (SEQ ID NO:1). The protein expressed from this plasmid has a Perfect DB sequence, a His tag sequence and a Factor Xa sequence. The amino acid sequence of the protein is shown in SEQ ID NO:12, and the nucleotide sequence is shown in SEQ ID NO:13.

(2) Expression and Purification as Well as Preparation of Samples Under Various Buffer Conditions

pCold08 hDi-R prepared in (1) above was used to transform Escherichia coli BL21. Transformants were grown on LB medium (containing 50 μg/ml of ampicillin) containing agar at a concentration of 1.5% (w/v). A grown colony was inoculated into 2.5 ml of LB liquid medium (containing 50 μg/ml of ampicillin) and cultured at 37° C. overnight. A portion of the culture was inoculated into 100 ml of LB medium and cultured at 37° C. until the logarithmic growth phase. After cultivation, the culture was shaken for 10 minutes in an incubator at 15° C., IPTG was added thereto at a final concentration of 1.0 mM, and the cultivation was continued at 15° C. for 24 hours for expression induction. The cells were collected by centrifugation, and resuspended in 5 ml of a cell disruption solution (50 mM tris-hydrochloride buffer (pH 7.5), 100 mM sodium chloride, 0.5 mM EDTA, 1% Triton X-100, 1 mM dithiothreitol, 2 mM phenylmethylsulfonyl fluoride). The cells disrupted by sonication were subjected to centrifugation (11,000 rpm, 20 minutes) to separate a supernatant extract from a precipitate.

About 5 ml of the supernatant extract was further purified using a nickel column as follows.

10 ml of buffer A (20 mM tris-hydrochloride buffer (pH 7.5), 100 mM sodium chloride, 1 mM dithiothreitol, 0.1% Triton X-100) was added to and mixed with Ni-NTA agarose (Qiagen) corresponding to a resin volume of 1 ml. The mixture was centrifuged at 1,500 rpm for several minutes. The supernatant was discarded and about 1 ml of the resin was collected. About 5 ml of the supernatant prepared from the disrupted cells was added to the resin, and mixed gently using a rotary shaker at 4° C. for about one hour. Then, the resin to which the protein of interest had been adsorbed was filled in a φ15-mm column, and washed twice with 5 ml of buffer A. The resin was then washed with 5 ml of buffer B (20 mM tris-hydrochloride buffer (pH 7.5), 100 mM sodium chloride, 1 mM dithiothreitol, 0.1% Triton X-100, 40 mM imidazole). The resin was further washed with 5 ml of buffer C (20 mM tris-hydrochloride buffer (pH 7.5), 800 mM sodium chloride, 1 mM dithiothreitol, 0.1% Triton X-100, 40 mM imidazole) followed by 5 ml of buffer B to remove unnecessary proteins other than the protein of interest.

After washing, elution was carried out with 3 ml of buffer D (20 mM tris-hydrochloride buffer (pH 7.5), 100 mM sodium chloride, 1 mM dithiothreitol, 0.1% Triton X-100, 100 mM imidazole). The eluate was dialyzed against 500 ml of buffer E (50 mM tris-hydrochloride buffer (pH 8.0), 100 mM sodium chloride, 0.5 mM EDTA, 0.1% Triton X-100, 1 mM dithiothreitol) and concentrated about 10-fold using Centricon (Amicon). When a portion of the purified and concentrated sample was subjected to electrophoresis on 10% SDS-polyacrylamide gel, a band for the protein of interest was observed at a position corresponding to a molecular weight of about 76,800. When Western blotting detection with an anti-His tag antibody was carried out for the sample using Anti His HRP Conjugate (Qiagen) according to the attached protocol, the band for the protein of interest was detected upon color development. Hereinafter, the human Dicer RNase III domain protein is called hDiR.

According to the above-mentioned method, the eluate with buffer D was dialyzed against buffer E. This sample is designated as Protein Sample I. Dialyses were also carried out using buffers having the following compositions in order to determine the conditions of protein shape buffer.

1. Dialysis using buffer F (50 mM tris-hydrochloride buffer (pH 8.0), 100 mM sodium chloride, 1 mM magnesium chloride, 0.1% Triton X-100, 1 mM dithiothreitol)→Protein Sample II.

2. Dialysis using buffer G (50 mM tris-hydrochloride buffer (pH 8.5), 100 mM sodium chloride, 1 mM magnesium chloride, 0.1% Triton X-100, 1 mM dithiothreitol)→Protein Sample III.

3. Dialysis using buffer H (50 mM tris-hydrochloride buffer (pH 8.8), 100 mM sodium chloride, 1 mM magnesium chloride, 0.1% Triton X-100, 1 mM dithiothreitol)→Protein Sample IV.

### Example 2

### Measurement of Activity of Degrading dsRNA

(1) Preparation of Reaction Mixture

Dicer activities of Protein Samples I to IV prepared in Example 1-(2) were measured. The activities were measured as follows.

A dsRNA as a substrate used for the activity measurements was synthesized using TurboScript T7 Transcription kit (GTS) according to the attached protocol.

Specifically, pDON-rsGFP was constructed by inserting a gene encoding red-shift green fluorescent protein (hereinafter referred to as GFP) (SEQ ID NO:11) from a plasmid pQBI125 (Wako Pure Chemical Industries) into a plasmid pDON-AI (Takara Bio). A PCR was carried out using pDON-rsGFP as a template as well as a synthetic primer 3 (SEQ ID NO:7) which has a T7 promoter sequence and a synthetic primer 4 (SEQ ID NO:8) to obtain an amplification product. An about 700-bp dsRNA was prepared by an RNA synthesis reaction using the resulting double-stranded DNA as a template and T7 RNA polymerase.

A reaction mixture of a total volume of 10 μl was prepared by adding 1 μg of the dsRNA prepared as described above, 1 μl of hDiR prepared in (2) above, 1 μl of 10 mM ATP solution, 1 μl of 50 mM magnesium chloride solution, 2 μl of 5× reaction buffer (a 5-fold concentrate of the buffer used for final dialysis) and nuclease-free water.

In case of a commercially available Dicer (GTS), a reaction mixture of a total volume of 10 μl was prepared by adding 2 μl of Dicer enzyme solution, 1 μg of the dsRNA as a substrate, 1 μl of 10 mM ATP solution, 0.5 μl of 50 mM magnesium chloride solution, 4 μl of the attached reaction buffer and nuclease-free water.

After the above-mentioned reaction mixtures were prepared and reacted at 37° C. for 17 hours, 5 μl each of the reaction mixtures was subjected to electrophoresis on 15% polyacrylamide gel and staining with ethidium bromide to observe cleavage products. A reaction mixture for which a degradation product of about 21 nucleotides was observed in the gel stained with ethidium bromide after electrophoresis was determined to have an activity.

(2) Activity Measurement

The activities of the reaction mixtures prepared in (1) above were measured to examine the influences on the RNase III domain protein (hDiR). Furthermore, samples immediately after the purification, or stored for five days at 4° C. or −20° C. were used in order to examine the stabilities in the buffers. As a result, the degradation products of about 21 nucleotides (the same size as that observed using the commercially available Dicer) were observed for Protein Sample III and Protein Sample IV stored either at 4° C. or at −20° C., demonstrating that the activities were stably retained.

Based on the above, it was shown that the activity of the RNase III domain protein of human Dicer (hDiR) can be stabilized by storing it in a tris-hydrochloride buffer at pH 8.5 or above in the presence of magnesium chloride.

### Example 3

### Examination of Factors that Contribute to Production and Degradation of dsRNA

(1) A protein having an activity of binding to a nucleic acid in a normal temperature range was examined in order to examine factors that contribute to production and degradation of a dsRNA.

It was difficult to obtain such a protein having an activity of binding to a nucleic acid. Then, a CspB protein from Thermotoga maritima having the amino acid sequence of SEQ ID NO:9 was used as a model protein. The protein was prepared according to the method as described in Protein Science, 8:394-403 (1999).

(2) Effect of CspB from Thermotoga maritima on dsRNA Degradation

An activity of degrading a dsRNA with the addition of CspB was measured as follows.

Briefly, when hDi-R was used as an enzyme, a reaction mixture of a total volume of 10 μl was prepared by adding 1 μl of hDiR (enzyme solution) prepared in Example 1-(2), 1 μl of a CspB solution prepared in (1) above, 1 μg of the dsRNA used as a substrate in Example 2-(1), 1 μl of 10 mM ATP solution, 1 μl of 50 mM magnesium chloride solution, 2 μl of 5× reaction buffer (250 mM tris-hydrochloride buffer (pH 8.5), 500 mM sodium chloride, 0.5% Triton X-100, 5 mM DTT) and nuclease-free water. In case of a commercially available Dicer, a reaction mixture of a total volume of 10 μl was prepared by adding 1 μg of the dsRNA as a substrate and nuclease-free water to the composition as described in the attached instructions. One from GTS, Stratagene or Invitrogen was used as a commercially available Dicer.

The CspB protein was added at a final concentration of 4.6 ng/μl, 9.2 ng/μl, 18.4 ng/μl or 92 ng/μl. 1 μl of 10 mM potassium phosphate buffer (pH 7.5) as a shape buffer for CspB was added to a control (no addition).

After the above-mentioned reaction mixtures were prepared and reacted at 37° C. for 18 hours, 5 μl each of the reaction mixtures was subjected to electrophoresis on 15% polyacrylamide gel and staining with ethidium bromide to observe degradation products. Furthermore, the gel was subjected to image analysis using Total Lab ver. 1.11 (Nonlinear Dynamics) to quantify dsRNA degradation products of about 21 nucleotides. As a result, increase in the amount of degraded dsRNA due to the addition of CspB was observed using each amount of the commercially available Dicer or the RNase III domain protein (hDiR). In particular, increased degradation was observed around the concentration of 9.2 ng/μl.

Thus, it was shown that a more amount of a dsRNA degradation product can be obtained by adding CspB to a reaction mixture using any of the commercially available Dicers and the RNase III domain protein.

(3) Effect of CspB from Thermotoga maritima on RNA Synthesis

In RNA interference, it is also important to promote an activity of an RNA synthesis system for synthesis of a dsRNA. From this point of view, the effect of CspB from Thermotoga maritima on RNA synthesis was examined. A T7 RNA polymerase system was chosen as a model system. Influence on an RNA synthesis system was examined as follows. Briefly, an amount of transcript obtained using T7 RNA polymerase with the addition of CspB was used as an index. A plasmid was constructed by incorporating the rsGFP gene having the nucleotide sequence of SEQ ID NO:11 into pET16b (Novagen) having the T7 promoter which had been prepared according to a conventional method. A reaction mixture of a total volume of 20 μl was prepared by adding 1 μg of the constructed plasmid as a template DNA, 2 μl of 10×T7 RNA polymerase buffer (Takara Bio), 2 μl of 50 mM DTT, 0.4 μl of RNase inhibitor (Takara Bio), 2 μl of 25 mM NTPs, 1 μl of T7 RNA polymerase (Takara Bio), 1 μl of the CspB solution and nuclease-free water. The reaction mixture was reacted at 37° C. for 4 hours. The CspB protein was added at a final concentration of 90 ng/μl, 180 ng/μl, 460 ng/μl or 920 ng/μl in this Example. 1 μl of 10 mM potassium phosphate buffer (pH 7.5) as a shape buffer for CspB was added to a control (no addition).

2 μl of 10×MOPS buffer (200 mM MOPS, 50 mM sodium acetate, 1 mM EDTA, 10 mM EGTA), 2 μl of formaldehyde, 9 μl of deionized formamide and 3 μl of nuclease-free water were added to 2 μl of the reacted sample. The mixture was incubated at 70° C. for 10 minutes and then on ice for 1 minute. 2 μl of 10× loading buffer was added thereto, and the resulting mixture was subjected to electrophoretic analysis using 1.25% agarose gel containing ethidium bromide in 1×MOPS buffer. The gel was subjected to image analysis using Total Lab ver. 1.11 (Nonlinear Dynamics) to quantify an mRNA synthesized using T7 RNA polymerase. As a result, increase in the amount of transcript was observed for each concentration. In particular, it was confirmed that the amount of transcript in case of addition of CspB at 460 ng/μl or more was about 2-fold or more as compared with that in case of no addition, and that the amount in case of addition at 920 ng/μl was about 3-fold larger.

(4) Effect of CspB from Thermotoga maritima on dsRNA Synthesis

The influence of CspB from Thermotoga maritima on dsRNA synthesis was examined. The template for dsRNA synthesis prepared in Example 2-(1) was utilized in this Example. A commercially available product TurboScript T7 Transcription kit (Gene Therapy Systems) was used for transcription into RNA according to the attached protocol. The CspB protein was added at a final concentration of 90 ng/μl, 180 ng/μl, 460 ng/μl or 920 ng/μl. 1 μl of 10 mM potassium phosphate buffer (pH 7.5) as a shape buffer for CspB was added to a control (no addition). After reaction at 37° C. for 4 hours, 1 μl of DNase I was added thereto, and the reaction was continued at 37° C. for 15 minutes. 1 μl of a 40-fold dilution of the reaction mixture was subjected to electrophoresis on 1% agarose gel containing ethidium bromide. The gel was subjected to quantification of the dsRNA synthesized with T7 RNA polymerase according to the method as described in (3) above. As a result, increase in the amount of transcript was observed for each concentration. In particular, it was confirmed that the amount of transcript in case of addition of CspB at 920 ng/μl or more was about 2-fold or more as compared with that in case of no addition.

Another embodiment of T7 RNA polymerase transcription system was also examined in a manner similar to that in the above-mentioned Example. Specifically, a reaction mixture of a total volume of 10 μl was prepared by adding 1 μg of the template for dsRNA synthesis prepared in Example 2-(1), 1 μl of 10×T7 RNA polymerase buffer (Takara Bio), 1 μl of 50 mM DTT, 0.2 μl of RNase inhibitor (Takara Bio), 1 μl of 25 mM NTPs, 0.5 μl of T7 RNA polymerase (Takara Bio), 1 μl of the CspB solution and nuclease-free water. The reaction mixture was reacted at 37° C. for 4 hours. The CspB protein was added at a final concentration of 90 ng/μl, 180 ng/μl, 460 ng/μl or 920 ng/μl. 1 μl of 10 mM potassium phosphate buffer (pH 7.5) as a shape buffer for CspB was added to a control (no addition). After reaction, 0.5 μl of DNase I (Takara Bio) was added to the sample, and the reaction was continued at 37° C. for 15 minutes. 1 μl of a 40-fold dilution of the reaction mixture was subjected to electrophoresis on 1% agarose gel containing ethidium bromide. The gel was also subjected to quantification of the dsRNA. As a result, increase in the amount of transcript was observed for each concentration. In particular, it was confirmed that the amount of transcript in case of addition of CspB at 920 ng/μl or more was about 3-fold or more as compared with that in case of no addition.

As described above, it was shown that a more amount of transcript with T7 RNA polymerase can be obtained by adding CspB into a reaction mixture. This effect was observed upon synthesis of both a single-stranded RNA and a double-stranded RNA.

### Example 4

### Expression of PAZ+RNase III Domain of Human Dicer

(1) Construction of Expression Vector

An expression vector was constructed as follows in order to express a polypeptide consisting of amino acid 679 to amino acid 1924 (nucleotide 2035 to nucleotide 5772) in the N-terminal portion of the amino acid sequence of human Dicer as shown in SEQ ID NO:1.

First, synthetic primers 5 and 6 (SEQ ID NOS:14 and 15) were synthesized using a DNA synthesizer based on the nucleotide sequence available to the public under GenBank accession no. AB028449, and purified according to a conventional method. The synthetic primer 5 is a synthetic DNA that has a recognition sequence for a restriction enzyme KpnI at nucleotide 9 to nucleotide 14, and a nucleotide sequence corresponding to amino acid 679 to amino acid 685 in the amino acid sequence of human Dicer (SEQ ID NO:1) at nucleotide 16 to nucleotide 36. The synthetic primer 6 has a recognition sequence for a restriction enzyme HindIII at nucleotide 9 to nucleotide 14 and a nucleotide sequence corresponding to amino acid 1919 to amino acid 1924 in the amino acid sequence of human Dicer (SEQ ID NO:1) at nucleotide 18 to nucleotide 36.

A PCR was conducted using the synthetic primers. The reaction conditions for the PCR were as follows.

Briefly, a reaction mixture of a total volume of 50 μl was prepared by adding 2 μl of a template DNA (human cDNA library, human pancreas, Takara Bio), 5 μl of 10×LA PCR buffer (Takara Bio), 5 μl of dNTPs (Takara Bio), 10 pmol of the synthetic primer 5, 10 pmol of the synthetic primer 6, 0.5 U of Takara LA Taq (Takara Bio) and sterile water. The reaction mixture was placed in TaKaRa PCR Thermal Cycler SP (Takara Bio) and subjected to a reaction as follows: 30 cycles of 94° C. for 1 minute, 55° C. for 1 minute and 72° C. for 3 minutes.

After reaction, 5 μl of the reaction mixture was subjected to electrophoresis on 1.0% agarose gel. The observed about 2.7-kbp DNA fragment of interest was recovered and purified from the electrophoresis gel and subjected to ethanol precipitation. After ethanol precipitation, the recovered DNA was suspended in 5 μl of sterile water, and doubly digested with restriction enzymes KpnI (Takara Bio) and HindIII (Takara Bio). The KpnI-HindIII digest was extracted and purified after electrophoresis on 1.0% agarose gel to obtain a KpnI-HindIII-digested DNA fragment.

Next, the vector pCold08NC2 prepared in Example 1-(1) was cleaved with the same restriction enzymes as those used upon preparation of the KpnI-HindIII-digested DNA fragment and the termini were dephosphorylated. The thus prepared vector and the KpnI-HindIII-digested DNA fragment were mixed together and ligated to each other using DNA ligation kit (Takara Bio). 20 μl of the ligation mixture was used to transform Escherichia coli JM109. Transformants were grown on LB medium (containing 50 μg/ml of ampicillin) containing agar at a concentration of 1.5% (w/v).

A plasmid having the inserted DNA fragment of interest was confirmed by sequencing. This recombinant plasmid was designated as pCold08 hDi-ASI. This plasmid is designated and indicated as pCold08 hDi-ASI and has been deposited under accession number FERM BP-10076 at International Patent Organism Depositary, National Institute of Advanced Industrial Science and Technology, AIST Tsukuba Central 6, 1-1, Higashi 1-chome, Tsukuba-shi, Ibaraki 305-8566, Japan since Sep. 26, 2003 (date of original deposit). pCold08 hDi-ASI is a plasmid containing a nucleotide sequence that encodes an amino acid sequence from amino acid 679 to amino acid 1924 in the amino acid sequence of human Dicer (SEQ ID NO:1) (the nucleotide sequence of SEQ ID NO:16, the amino acid sequence of SEQ ID NO:17). The protein expressed from this plasmid has a Perfect DB sequence, a His tag sequence and a Factor Xa sequence. The amino acid sequence of the protein is shown in SEQ ID NO:18, and the nucleotide sequence is shown in SEQ ID NO:19.

(2) Expression and Purification

pCold08 hDi-ASI prepared in (1) above was used to transform Escherichia coli BL21-CodonPlus-RIL strain (Stratagene). Transformants were grown on LB medium (containing 50 μg/ml of ampicillin) containing agar at a concentration of 1.5% (w/v). A grown colony was inoculated into 2.5 ml of LB liquid medium (containing 50 μg/ml of ampicillin) and cultured at 37° C. overnight. A portion of the culture was inoculated into 100 ml of LB medium and cultured at 37° C. until the logarithmic growth phase. After cultivation, the culture was shaken for 10 minutes in an incubator at 15° C., IPTG was added thereto at a final concentration of 1.0 mM, and the cultivation was continued at 15° C. for 24 hours for expression induction. The cells were collected by centrifugation, and resuspended in 5 ml of a cell disruption solution (50 mM tris-hydrochloride buffer (pH 8.5), 100 mM sodium chloride, 1 mM magnesium chloride, 0.1% Triton X-100, 1 mM dithiothreitol, 1 mM phenylmethylsulfonyl fluoride). The cells disrupted by sonication were subjected to centrifugation (11,000 rpm, 20 minutes) to separate a supernatant extract from a precipitate.

About 5 ml of the supernatant extract was further purified using a nickel column as follows.

10 ml of buffer A (20 mM tris-hydrochloride buffer (pH 8.5), 100 mM sodium chloride, 1 mM dithiothreitol, 1 mM magnesium chloride, 0.1% Triton X-100) was added to and mixed with Ni-NTA agarose (Qiagen) corresponding to a resin volume of 1 ml. The mixture was centrifuged at 1,500 rpm for several minutes. The supernatant was discarded and about 1 ml of the resin was collected. About 5 ml of the supernatant prepared from the disrupted cells was added to the resin, and mixed gently using a rotary shaker at 4° C. for about one hour. Then, the resin to which the protein of interest had been adsorbed was filled in a φ15-mm column, and washed twice with 5 ml of buffer A. The resin was then washed with 5 ml of buffer B (20 mM tris-hydrochloride buffer (pH 8.5), 100 mM sodium chloride, 1 mM magnesium chloride, 1 mM dithiothreitol, 0.1% Triton X-100, 40 mM imidazole). The resin was further washed with 5 ml of buffer C (20 mM tris-hydrochloride buffer (pH 8.5), 800 mM sodium chloride, 1 mM magnesium chloride, 1 mM dithiothreitol, 0.1% Triton X-100, 40 mM imidazole) followed by 5 ml of buffer B to remove unnecessary proteins other than the protein of interest.

After washing, elution was carried out with 3 ml of buffer D (20 mM tris-hydrochloride buffer (pH 8.5), 100 mM sodium chloride, 1 mM magnesium chloride, 1 mM dithiothreitol, 0.1%-Triton X-100, 100 mM imidazole). The eluate was dialyzed against 500 ml of buffer E (50 mM tris-hydrochloride buffer (pH 8.5), 100 mM sodium chloride, 1 mM magnesium chloride, 0.1% Triton X-100, 1 mM dithiothreitol) and concentrated about 10-fold using Centricon (Amicon). When a portion of the concentrate was subjected to electrophoresis on 10% SDS-polyacrylamide gel, a band for the protein of interest was observed at a position corresponding to a molecular weight of about 144,000 for a sample obtained using Escherichia coli BL21-CodonPlus-RIL strain (Stratagene) as a host. This sample was used for subsequent confirmation of the activity. Hereinafter, the human Dicer PAZ+RNase III domain protein is called hDi-ASI.

(3) Measurement of Activity of Degrading dsRNA

The activities of degrading a dsRNA of the protein samples prepared in Example 4-(2) above were measured according to the method as described in Example 2.

As a result, a degradation product of about 21 nucleotides was observed in the gel stained with ethidium bromide after electrophoresis. Thus, activities of degrading a dsRNA were observed for the RNase III domain protein (hDiR) and the protein containing the PAZ region (hDi-ASI).

Furthermore, the protein sample was subjected to 6 or 10 cycles of freezing at −80° C. and thawing at room temperature in order to examine the freeze-thaw stability. As a control, hDiR prepared in Example 1-(2) was examined in a similar manner.

As a result, the PAZ+RNase III domain protein (hDi-ASI) retained the degradation activity after 6 or 10 freeze-thaw cycles. On the other hand, the activity of hDiR was observed after up to 6 cycles of freeze-thaw cycles. Based on these results, it was confirmed that the protein acquired a more freeze-thaw stability by including the PAZ domain in addition to the RNase III domain.

(4) Examination of Factors that Contribute to Degradation

Influence of a protein having an activity of binding to a nucleic acid in a normal temperature range was examined for hDi-ASI prepared in Example 4-(2).

The CspB protein from Thermotoga maritima prepared in Example 3 was used as such a protein having an activity of binding to a nucleic acid. The effect on dsRNA degradation was determined as follows.

Briefly, a reaction mixture of a total volume of 10 μl was prepared by adding 1 μl of hDi-ASI (enzyme solution) prepared in Example 4-(2), 1 μl of the CspB solution prepared in Example 3, 1 μg of a dsRNA as a substrate, 1 μl of 10 mM ATP solution, 1 μl of 50 mM magnesium chloride solution, 2 μl of 5× reaction buffer (250 mM tris-hydrochloride (pH 8.5), 750 mM sodium chloride, 0.5% Triton X-100, 5 mM DTT) and nuclease-free water. The CspB protein was added at a final concentration of 9.2 ng/μl, 18.4 ng/μl or 92 ng/μl. 1 μl of 10 mM potassium phosphate buffer (pH 7.5) as a shape buffer for CspB was added to a control (no addition).

After the above-mentioned reaction mixtures were prepared and reacted at 37° C. for 17 hours, 5 μl each of the reaction mixtures was subjected to electrophoresis on 15% polyacrylamide gel and staining with ethidium bromide to observe cleavage products. Furthermore, the gel was subjected to image analysis using Total Lab ver. 1.11 (Nonlinear Dynamics) to quantify dsRNA degradation products of about 21 nucleotides.

As a result, increase in the amount of degraded dsRNA due to the addition of CspB was observed also in case of hDi-ASI. In particular, increased degradation was observed around the concentration of 9.2 ng/μl.

Thus, it was confirmed that CspB promoted dsRNA degradation activities of human Dicers (including native forms and mutant forms) containing the RNase III domain.

### Example 5

The RNA interference effect of an siRNA prepared using human-derived hDiR of the present invention was examined. A commercially available Dicer (GTS) was used as a control. dsRNA degradation products were prepared basically according to the method as described in Example 2-(1). Specifically, 10 μg of a dsRNA was cleaved at 37° C. for 18 hours using 10 units of hDiR as described in Example 1-(2) or the commercially available Dicer. The cleavage products were purified using RNA Purification Column 1, 2 (Gene Therapy Systems) and used for assessments in RNA interference as follows.

Briefly, an appropriate number (5×10⁴ cells) of 293 cells were seeded into a 24-well plate containing D-MEM medium (SIGMA) supplemented with 10% FBS and 1% penicillin/streptomycin 24 hours before transfer of an siRNA, and cultured overnight in a CO₂ incubator. The cells were cultured to about 80% confluence. 3 μl of TransIT 293 Transfection Reagent (Takara Bio) was added to 50 μl of a serum-free medium. The mixture was vigorously stirred and allowed to stand at room temperature for 5 minutes. 0.3 μg of pQBI25 (Wako Pure Chemical Industries) was added thereto, and the mixture was gently mixed and allowed to stand at room temperature for 5 minutes. 4 μl of TransIT-TKO reagent was added thereto, and the mixture was gently mixed and allowed to stand at room temperature for 5 minutes. 500 ng of the siRNA was added thereto, and the mixture was gently mixed and allowed to stand at room temperature for 5 minutes to obtain a DNA/siRNA solution. The DNA/siRNA solution was added dropwise to 250 μl of D-MEM medium supplemented with 10% FBS in each well, and the mixture was mixed gently so that the solution in the well became homogeneous. 0.3 μg of pQBI25 (Wako Pure Chemical Industries) or sterile water was added alone to controls. The cells were cultured for 24 hours in a CO₂ incubator. The cells were subjected to flow cytometry using FACS Vantage (Becton-Dickinson) to determine the inhibitory effect of the transferred DNA/siRNA solution on GFP expression as compared with the control with the vector (DNA) alone. The results are shown in Table 1.

TABLE 1

| | Average |
|---|---|
| | fluorescence |
| Transferred sample | intensity |
| Control (no addition) | 8.09 |
| Control (vector alone) | 1331.44 |
| hDiR | 14.92 |
| Commercially available Dicer | 71.57 |

A less average fluorescence value as compared with the control (vector alone) as shown in Table 1 represents more RNA interference. It was confirmed that the siRNA obtained using hDiR exhibited an RNA interference effect like the one obtained using the commercially available Dicer, and the exhibited RNA interference effect was stronger than that of the one obtained using the commercially available Dicer.

Based on the above, it was confirmed that hDiR of the present invention is useful for preparation of an siRNA for RNA interference.

### Example 6

The RNA interference effect of a varying amount of an siRNA prepared using hDiR of the present invention was examined. A commercially available Dicer (Gene Therapy Systems) was used as a control. Cleavage of a dsRNA was carried out basically according to the method as described in Example 2-(1). Specifically, 10 μg of a dsRNA was cleaved at 37° C. for 18 hours using 10 μl of hDiR as described in Example 1-(2) or the commercially available Dicer. CspB used in Example 3-(2) was added at a final concentration of 9.2 ng/μl and reacted.

The cleavage products were purified using RNA Purification Column 1, 2 (Gene Therapy Systems) and used for assessments in RNA interference as follows.

Briefly, an appropriate number (1.5×10⁵ cells) of 293 cells were seeded into a 24-well plate containing D-MEM medium (SIGMA) supplemented with 10% FBS and 1% penicillin/streptomycin 24 hours before transfer of an siRNA, and cultured overnight in a CO₂ incubator. The cells were cultured to about 95% confluence. 1 μl of Genejuice Transfection Reagent (Takara Bio) was added to 49 μl of a serum-free medium. The mixture was vigorously stirred and allowed to stand at room temperature for 5 minutes. 0.3 μg of pQBI25 (Wako Pure Chemical Industries) was added thereto, and the mixture was gently mixed and allowed to stand at room temperature for 5 minutes.

In parallel, 3 μl of Ribojuice Transfection Reagent (Takara Bio) was added to 47 up of a serum-free medium in another tube, and the mixture was vigorously stirred. After allowing to stand at room temperature for 5 minutes, 166.7 ng, 55.6 ng or 18.5 ng of the siRNA was added thereto, and the mixture was gently mixed and allowed to stand at room temperature for 5 minutes.

One of the two types of solutions prepared as described above was added dropwise to 250 μl of D-MEM medium supplemented with 10% FBS in each well, and the mixture was mixed gently so that the solution in the well became homogeneous. The vector (DNA) or sterile water was added alone to controls. The cells were cultured for 24 hours in a CO₂ incubator. The cells were subjected to flow cytometry using FACS Vantage (Becton-Dickinson) to determine the inhibitory effect of the transferred DNA/siRNA solution on rsGFP expression as compared with the control with the vector (DNA) alone. The results are shown in Table 2.

TABLE 2

| | Average |
|---|---|
| | fluorescence |
| | intensity |
| Transferred sample | (relative value) |
| Control (no addition) | 0 |
| Control (vector alone) | 100 |
| hDiR 166.7 ng | 18.21 |
| hDiR 55.6 ng | 18.97 |
| hDiR 18.5 ng | 32.67 |
| Commercially available Dicer 166.7 ng | 17.74 |
| Commercially available Dicer 55.6 ng | 19.80 |
| Commercially available Dicer 18.5 ng | 32.34 |

A less average fluorescence intensity value as compared with the control (vector alone) as shown in Table 2 represents more RNA interference. It was confirmed that the siRNA obtained using hDiR exhibited an RNA interference effect like the one obtained using the commercially available Dicer.

Based on the above, it was confirmed that hDiR of the present invention is useful for preparation of an siRNA for RNA interference.

Total RNAs extracted from the cell samples were subjected to real-time RT-PCR to quantify the mRNAs for rsGFP for assessment in RNA interference.

Specifically, a total RNA was extracted and purified from cells cultured for 24 hours at 37° C. in a CO₂ incubator after the transfer of an siRNA using trizole (Invitrogen) according to the attached protocol. A reaction mixture of a total volume of 20 μl was prepared by adding 80 ng of the total RNA, 4 μl of 5×M-MLV buffer (Takara Bio), 1 μl of 10 mM dNTPs (Takara Bio), 100 pmol of random hexamer, 20 U of RNase Inhibitor (Takara Bio), 100 U of M-MLV Reverse Transcriptase and sterile water. The reaction mixture was placed in TaKaRa PCR Thermal Cycler SP (Takara Bio) and subjected to a reaction at 42° C. for 10 minutes and then at 95° C. for 2 minutes. 20 μl of a reaction dilution solution (1×M-MLV buffer, 0.5 mM dNTP mixture) was added to the reaction mixture. 10 μl aliquots of the resulting mixture were dispensed. 2.5 μl of 10×R-PCR buffer (Takara Bio), 0.3 μl of 250 mM Mg²⁺, 0.75 μl of 10 mM dNTPs, 1.25 U of TaKaRa Ex Taq R-PCR (Takara Bio), 2.5 μl of a 3000-fold dilution of SYBR Green (Takara Bio) in sterile water and 1.25 μl of 100% DMSO were added to 10 μl of the reaction mixture. 5 pmol each of synthetic primers for detecting β-actin (Takara Bio), GAPDH (Takara Bio), rsGFP (rsGFP-F (SEQ ID NO:20), rsGFP-R (SEQ ID NO:21)) or Neo (Neo-F (SEQ ID NO:22), Neo-R (SEQ ID NO:23)) and sterile water to a total volume of 25 μl were further added. The reaction mixtures were placed in Smart Cycler II Unit (Takara Bio), heat-denatured at 95° C. for 10 seconds, and subjected to a reaction as follows: 45 cycles of 95° C. for 5 seconds and 60° C. for 20 seconds. The obtained data were analyzed to quantify the mRNAs for human β-actin and GAPDH as well as rsGFP and Neo from the transferred plasmid. The results are shown in Table 3.

TABLE 3

| | Amount of |
|---|---|
| | rsGFP mRNA |
| Transferred sample | (relative value) |
| Control (no addition) | 0 |
| Control (vector alone) | 100 |
| hDiR 166.7 ng | 15.92 |
| hDiR 55.6 ng | 22.72 |
| hDiR 18.5 ng | 30.61 |
| Commercially available Dicer 166.7 ng | 14.77 |
| Commercially available Dicer 55.6 ng | 30.91 |
| Commercially available Dicer 18.5 ng | 35.84 |

A less amount of rsGFP mRNA as compared with the control (vector alone) as shown in Table 3 represents more RNA interference. It was confirmed that hDiR resulted in an RNA interference effect like the commercially available Dicer.

Based on the above, it was confirmed that hDiR of the present invention is useful for preparation of an siRNA for RNA interference.

### Example 7

The samples prepared in Example 1-(2) and Example 4-(2) were assessed using a dsRNA as a substrate for measuring their dsRNA degradation activities which was prepared from a luciferase gene. A dsRNA was synthesized using TurboScript T7 Transcription kit (GTS) according to the attached protocol in a manner similar to that in Example 2-(1).

Specifically, a PCR was carried out using a plasmid pGL3-Basic vector (Promega) as a template as well as a primer dsl-1 (SEQ ID NO:24) which has a T7 promoter sequence and a primer dsl-2 (SEQ ID NO:25) to obtain the luciferase-encoding gene inserted into the plasmid pGL3-Basic vector (Promega) as an amplification product of about 500 base pairs. An about 500-bp dsRNA was prepared by an RNA synthesis reaction using the resulting double-stranded DNA as a template and T7 RNA polymerase.

The RNA interference effects of siRNAs prepared using hDiR or hDi-ASI of the present invention were examined. A commercially available Dicer (Gene Therapy Systems) was used as a control. Cleavage of a dsRNA was carried out basically according to the method as described in Example 2-(1). Specifically, 10 μg of the dsRNA was cleaved at 37° C. for 18 hours using 10 μl of hDiR as described in Example 1-(2), hDi-ASI as described in Example 4-(2) or the commercially available Dicer. CspB used in Example 3-(2) was added at a final concentration of 9.2 ng/μl and reacted.

The cleavage products were purified using RNA Purification Column 1, 2 (Gene Therapy Systems) and used for assessments in RNA interference as follows.

Briefly, an appropriate number (1.5×10⁵ cells) of 293 cells were seeded into a 24-well plate containing D-MEM medium (SIGMA) supplemented with 10% FBS and 1% penicillin/streptomycin 24 hours before transfer of an siRNA, and cultured overnight in a CO₂ incubator. The cells were cultured to about 95% confluence. 1 μl of Genejuice Transfection Reagent (Takara Bio) was added to 49 μl of a serum-free medium. The mixture was vigorously stirred and allowed to stand at room temperature for 5 minutes. 0.5 μg of pGL3-control and 0.1 μg pRL-TK (Promega) were added thereto, and the mixture was gently mixed and allowed to stand at room temperature for 5 minutes.

In parallel, 3 μl of Ribojuice Transfection Reagent (Takara Bio) was added to 47 μl of a serum-free medium in another tube, and the mixture was vigorously stirred. After allowing to stand at room temperature for 5 minutes, 166.7 ng, 55.6 ng or 18.5 ng of the siRNA was added thereto, and the mixture was gently mixed and allowed to stand at room temperature for 5 minutes.

One of the two types of solutions prepared as described above was added dropwise to 250 μl of D-MEM medium supplemented with 10% FBS in each well, and the mixture was mixed gently so that the solution in the well became homogeneous. The vector (DNA) or sterile water was added alone to controls. The cells were cultured for 24 hours in a CO₂ incubator. The cells were subjected to an assay using Dual Luciferase Reporter assay kit (Promega) to determine the inhibitory effect of the addition of the siRNA on GL3 protein expression as compared with the control with the vector (DNA) alone. The results are shown in Table 4.

TABLE 4

| | GL3 |
|---|---|
| | expression level |
| Transferred siRNA sample | (relative value) |
| Control (no addition) | 0 |
| Control (vector alone) | 100 |
| hDiR 166.7 ng | 25.70 |
| hDiR 55.6 ng | 38.90 |
| hDiR 18.5 ng | 74.51 |
| hDi-ASI 166.7 ng | 25.87 |
| hDi-ASI 55.6 ng | 25.00 |
| hDi-ASI 18.5 ng | 31.09 |
| Commercially available Dicer 166.7 ng | 25.11 |
| Commercially available Dicer 55.6 ng | 30.15 |
| Commercially available Dicer 18.5 ng | 55.41 |

It is considered that a less GL3 expression level as compared with the control (vector alone) as shown in Table 4 represents more RNA interference. It was confirmed that the siRNA obtained using hDiR or hDi-ASI exhibited an RNA interference effect like the one obtained using the commercially available Dicer.

Based on the above, it was confirmed that hDiR and hDi-ASI of the present invention are useful for preparation of an siRNA for RNA interference.

### Example 8

The RNA interference effects of siRNAs prepared using hDiR or hDi-ASI of the present invention with or without the addition of CspB were examined. The procedure followed the method as described in Example 7 using luciferase. A commercially available Dicer (Gene Therapy Systems) was used as a control. Cleavage of a dsRNA was carried out basically according to the method as described in Example 2-(1) Specifically, 10 μg of the dsRNA was cleaved at 37° C. for 18 hours using 10 μl of hDiR prepared as described in Example 1-(2), hDi-ASI prepared as described in Example 4-(2) or the commercially available Dicer. The reactions were carried out with or without the addition of CspB used in Example 3-(2) at a final concentration of 9.2 ng/μl.

The cleavage products were purified using RNA Purification Column 1, 2 (Gene Therapy Systems) and used for assessments in RNA interference as follows.

TABLE 5

| | GL3 |
|---|---|
| | expression level |
| Transferred siRNA sample | (relative value) |
| Control (no addition) | 0 |
| Control (vector alone) | 100 |
| hDiR 55.6 ng + CspB | 9.66 |
| hDiR 55.6 ng | 16.40 |
| hDi-ASI 55.6 ng + CspB | 14.31 |
| hDi-ASI 55.6 ng | 13.19 |
| Commercially available Dicer 55.6 ng + CspB | 10.40 |
| Commercially available Dicer 55.6 ng | 12.07 |

It is considered that a less GL3 expression level as compared with the control (vector alone) as shown in Table 5 represents more RNA interference. It was shown that there was no difference in RNA interference effect between samples with or without the addition of CspB.

Based on the above, it was confirmed that CspB of the present invention does not influence the quality of an siRNA for RNA interference upon preparation of the siRNA.

### Example 9

### Stability of Enzyme

The PAZ domain+RNase III domain protein (hDi-ASI) purified in Example 4-(2) was stored, with the addition of CspB at a final concentration of 92 ng/μl, in a storage buffer I (50 mM tris-hydrochloride buffer (pH 8.5), 250 mM sodium chloride, 1 mM magnesium chloride, 0.1 mM DTT, 0.1% Triton X-100, 50% glycerol). Samples were-taken at an interval and their dsRNA degradation activities were measure as follows. A reaction mixture of a total volume of 10 μl was prepared by adding 1 μg of the dsRNA prepared in Example 2-(1), 1 μl of a sample from the protein prepared in 4-(2) above, 2 μl of 5× reaction buffer (100 mM tris-hydrochloride buffer (pH 8.5), 750 mM sodium chloride, 12.5 mM magnesium chloride) and nuclease-free water. After the reaction mixture was reacted at 37° C. for 18 hours, 5 μl of the reaction mixture was subjected to electrophoresis on 15% polyacrylamide gel and staining with ethidium bromide to observe the cleavage product. As a result, a degradation product of about 21 base pairs was observed for a sample stored for 6 months or longer, indicating its dsRNA degradation activity. The activity of the RNase III domain protein (hDiR) stored in the same buffer with the addition of CspB was retained for 3 months or more.

### Example 10

### Construction of pCold14-TmCspB Cultivation of Recombinant, and Purification

(1) Construction of pCold14-TmCspB

CspB from Thermotoga maritima (hereinafter referred to as TmCspB) was cloned as follows referring to the sequences as described in Protein Science, 8:394-403 (1999). The amino acid sequence and the nucleotide sequence are shown in SEQ ID NO:9 and SEQ ID NO:10, respectively.

First, synthetic primers E and F (SEQ ID NOS:26 and 27) were synthesized using a DNA synthesizer, and purified according to a conventional method. The synthetic primer E is a synthetic DNA that has a recognition sequence for a restriction enzyme NdeI at nucleotide 11 to nucleotide 16, and a nucleotide sequence corresponding to amino acid 1 to amino acid 7 in the amino acid sequence of TmCspB (SEQ ID NO:26) at nucleotide 14 to nucleotide 34. The synthetic primer F is a synthetic DNA that has a recognition sequence for a restriction enzyme BamHI at nucleotide 11 to nucleotide 16 and a nucleotide sequence corresponding to amino acid 61 to amino acid 66 in the amino acid sequence of TmCspB (SEQ ID NO:26) at nucleotide 20 to nucleotide 37.

A PCR was conducted using the synthetic primers. The reaction conditions for the PCR were as follows.

Briefly, a reaction mixture of a total volume of 50 μl was prepared by adding 1 μl (50 ng) of the template DNA as described in Example 3-(1), 5 μl of 10× Ex Taq buffer (Takara Bio), 5 μl of dNTPs (Takara Bio), 10 pmol of the synthetic primer E, 10 pmol of the synthetic primer F, 0.5 U of Takara Ex Taq (Takara Bio) and sterile water. The reaction mixture was placed in TaKaRa PCR Thermal Cycler SP (Takara Bio) and subjected to a reaction as follows: 30 cycles of 94° C. for 1 minute, 55° C. for 1 minute and 72° C. for 1 minute.

After reaction, 5 μl of the reaction mixture was subjected to electrophoresis on 3.0% agarose gel, and an about 220-bp DNA fragment of interest was observed. The remaining PCR reaction mixture was subjected to electrophoresis, and the fragment was recovered, purified and subjected to ethanol precipitation. After ethanol precipitation, the recovered DNA was suspended in 5 μl of sterile water, and doubly digested with restriction enzymes NdeI (Takara Bio) and BamHI (Takara Bio). The NdeI-BamHI digest was extracted and purified after electrophoresis on 3.0% agarose gel to obtain a NdeI-BamHI-digested DNA fragment.

Next, a vector pCold04NC2 was prepared according to the method described in Examples 1-6 of WO 99/27117 (hereinafter, the vector pCold04NC2 is called a vector pCold14).

The vector pCold14 was cleaved with the same restriction enzymes as those used upon preparation of the above-mentioned DNA fragment and the termini were dephosphorylated. The thus prepared vector and the NdeI-BamHI-digested DNA fragment were mixed together and ligated to each other using DNA ligation kit (Takara Bio) 20 μl of the ligation mixture was used to transform Escherichia coli JM109. Transformants were grown on LB medium (containing 50 μg/ml of ampicillin) containing agar at a concentration of 1.5% (w/v).

A plasmid having the inserted DNA fragment of interest was confirmed by sequencing.

(2) Cultivation in 5-Liter Jar and Purification of CspB

pCold14-TmCspB prepared in Example 10-(1) above was used to transform Escherichia coli BL21. Transformants were grown on LB medium (containing 50 μg/ml of ampicillin) containing agar at a concentration of 1.5% (w/v). A grown colony was inoculated into 30 ml of LB liquid medium (0.3 g of bacto-tryptone, 0.15 g of bacto-yeast extract, 0.15 g of NaCl, 1.5 mg of ampicillin) and cultured at 37° C. overnight. 30 ml of the culture was inoculated into 3 liters of LB liquid medium (30 g of bacto-tryptone, 15 g of bacto-yeast extract, 15 g of NaCl, 150 mg of ampicillin) in a 5-liter jar fermentor (Able) and cultured at 37° C. at 150 rpm with aeration at 0.5 liter/minute until the logarithmic growth phase. Thereafter, the culture was cooled to 15° C. After cooling, IPTG was added thereto at a final concentration of 1.0 mM, and the cultivation was continued at 15° C. at 150 rpm with aeration at 0.5 liter/minute for 24 hours for expression induction. The cells were collected by centrifugation to obtain 11 g of wet cells. The wet cells (11 g) were resuspended in 44 ml of buffer A (20 mM tris-hydrochloride buffer (pH 8.0)). The cells disrupted by sonication were subjected to centrifugation (10,000×g, 20 minutes) to separate a supernatant extract from a precipitate. The supernatant extract was further subjected to ultracentrifugation (70,000×g, 20 minutes) to separate a supernatant extract from a precipitate.

About 53 ml of the supernatant extract was heated in a water bath (70° C., 10 minutes), and centrifuged (10,000×g, 20 minutes) to separate a supernatant from a precipitate. NaCl at a final concentration of 200 mM was added to the heated supernatant. 10 ml of AnionExchanger DE52 equilibrated with buffer B (20 mM tris-hydrochloride buffer (pH 8.0), 200 mM sodium chloride) was further added thereto. The resulting mixture was gently stirred at 4° C. overnight. The mixture was centrifuged (3,000×g, 20 minutes) to separate a supernatant from a precipitate. The supernatant was filtrated through a glass filter. 45 ml of the filtrate was dialyzed against 3 liters of buffer A (20 mM tris-hydrochloride buffer (pH 8.0)).

50 ml of the dialysate was added to Q-Sepharose F.F. (Amersham Biosciences) corresponding to a resin volume of 100 ml filled in a φ20-mm column. The column was washed with 400 ml of buffer A (20 mM tris-hydrochloride buffer (pH 8.0)). A fraction not adsorbed to the column was collected and passed through UF 30,000-cut Centriprep YM-30 (Millipore). 260 ml of the UF-passed solution was added to 40 ml of Heparin Sepharose CL-6B (Amersham Biosciences) filled in a φ30-mm column. The column was washed with 160 ml of buffer A (20 mM tris-hydrochloride buffer (pH 8.0)). The protein of interest was eluted with a gradient of 0 to 1 M sodium chloride in 200 ml of buffer A (20 mM tris-hydrochloride buffer (pH 8.0)). A fraction with which the protein of interest having a molecular weight of about 7,500 was observed upon tricine-SDS-polyacrylamide gel electrophoresis was collected, and dialyzed against 3 liters of buffer A (20 mM tris-hydrochloride buffer (pH 8.0)). 65 ml of the dialysate was concentrated about 144-fold using UF 3,000-cut Centriprep YM-3 (Millipore) to obtain an about 450 μl of a protein sample. An equal amount of glycerol was added to the protein sample to obtain 850 μl of a 50% (w/w) glycerol solution. A portion of the solution was subjected to tricine-SDS-polyacrylamide gel electrophoresis, and a single stained band for the protein of interest was observed at a position corresponding to a molecular weight of about 7,500.

(3) Increase in Cleavage Activity by CspB Protein

Influence of a protein having an activity of binding to a nucleic acid (CspB) was examined using the protein samples prepared in Example 1-(2) and Example 4-(2). In this case, TmCspB prepared in Example 10-(2) was used.

Specifically, a reaction mixture of a total volume of 10 μl was prepared by adding 1 μl of the protein sample prepared in Example 1-(2) or Example 4-(2) (hDi-R enzyme solution or hDi-ASI enzyme solution), 1 μl of the CspB solution (92 ng/μl) prepared in Example 10-(2), 1 μg of the dsRNA as a substrate prepared in Example 2-(1), 2 μl of 5× reaction buffer (100 mM tris-hydrochloride buffer (pH 8.5), 750 mM sodium chloride, 12.5 mM magnesium chloride) and nuclease-free water. The final concentration of the CspB protein was 9.2 ng/μl. A similar procedure was carried out using a commercially available Dicer as a control. In case of the commercially available Dicer (GTS), a reaction mixture of a total volume of 10 μl was prepared by adding 2 μl of an enzyme solution, 1 μl of the CspB solution, 1 μg of the dsRNA as a substrate, 1 μl of 10 mM ATP solution, 0.5 μl of 50 mM magnesium chloride solution, 4 μl of the attached reaction buffer and nuclease-free water. 1 μl of nuclease-free water was added in place of TmCspB for a negative control.

After the above-mentioned reaction mixtures were prepared and reacted at 37° C. for 18 hours, 5 μl each of the reaction mixtures was subjected to electrophoresis on 15% polyacrylamide gel and staining with ethidium bromide to observe cleavage products. The gel was subjected to image analysis using Total Lab ver. 1.11 (Nonlinear Dynamics) to quantify dsRNA degradation products of about 21 nucleotides.

As a result, increase in the amount of degraded dsRNA observed using TmCspB expressed and purified in Example 3-(1) was also observed using TmCspB expressed and purified in Example 10-(2).

### Example 11

### Cultivation in 30-Liter Jar and Purification of hDi-ASI

(1) Expression and Purification

pCold08 hDi-ASI prepared in Example 4-(1) was used to transform Escherichia coli BL21. Transformants were grown on LB medium (containing 50 μg/ml of ampicillin) containing agar at a concentration of 1.5% (w/v). A grown colony was inoculated into 200 ml of TB liquid medium (2.4 g of bacto-tryptone, 4.8 g of bacto-yeast extract, 0.8 ml of glycerol, 17 mM KH₂PO₄, 72 mM K₂HPO₄, 10 mg of ampicillin) and cultured at 37° C. overnight. 200 ml of the culture was inoculated into 20 liters of TB liquid medium (240 g of bacto-tryptone, 480 g of bacto-yeast extract, 80 ml of glycerol, 17 mM KH₂PO₄, 72 mM K₂HPO₄, 1 g of ampicillin) in a 30-liter jar fermentor (B. E. Marubishi) and cultured at 37° C. at 100 rpm with aeration at 6 liter/minute until the logarithmic growth phase. Thereafter, the culture was cooled to 15° C. After cooling, IPTG was added thereto at a final concentration of 1.0 mM, and the cultivation was continued at 15° C. at 100 rpm with aeration at 6 liter/minute for 24 hours for expression induction. The cells were collected by centrifugation to obtain 26 g of wet cells. A portion of the wet cells (12 g) were resuspended in 48 ml of cell disruption solution (50 mM tris-hydrochloride buffer (pH 8.5), 100 mM sodium chloride, 1 mM magnesium chloride, protease inhibitor (Complete, EDTA-free, Boehringer Mannheim)). The cells disrupted by sonication were subjected to centrifugation (12,000 rpm, 30 minutes) to separate a supernatant extract from a precipitate.

About 56 ml of the supernatant extract was further purified using a nickel column as follows.

50 ml of the cell disruption solution was added to and mixed with Ni-NTA agarose (Qiagen) corresponding to a resin volume of 16 ml. The mixture was centrifuged at 1,500 rpm for several minutes. The supernatant was discarded. This procedure was repeated twice, and about 8 ml of the resin was collected. About 56 ml of the supernatant prepared from the disrupted cells was added to the resin, and mixed gently using a rotary shaker at 4° C. for about one hour. Then, the resin to which the protein of interest had been adsorbed was filled in a φ20-mm column, and washed with 40 ml of a cell disruption solution (50 mM tris-hydrochloride buffer (pH 8.5), 100 mM sodium chloride, 1 mM magnesium chloride, protease inhibitor (Complete, EDTA-free, Boehringer Mannheim)). The resin was then washed with 40 ml of buffer A (20 mM tris-hydrochloride buffer (pH 8.5), 100 mM sodium chloride, 1 mM magnesium chloride, 10% glycerol, 20 mM imidazole), 40 ml of buffer B (20 mM tris-hydrochloride buffer (pH 8.5), 800 mM sodium chloride, 1 mM magnesium chloride, 10% glycerol, 20 mM imidazole), and 40 ml of buffer A to remove unnecessary proteins other than the protein of interest.

After washing, elution was carried out with 24 ml of buffer C (20 mM tris-hydrochloride buffer (pH 8.5), 100 mM sodium chloride, 1 mM magnesium chloride, 10% glycerol, 100 mM imidazole). The eluate was dialyzed against 300 ml of buffer D (20 mM tris-hydrochloride buffer (pH 8.5), 100 mM sodium chloride, 1 mM magnesium chloride, 10% glycerol).

The dialyzed enzyme solution was added to 1 ml of Heparin Sepharose CL-6B (Amersham Biosciences) filled in a φ10-mm column. The column was washed with 5 ml of buffer D (20 mM tris-hydrochloride buffer (pH 8.5), 100 mM sodium chloride, 1 mM magnesium chloride, 10% glycerol). Elution of protein was carried out using 5 ml of buffer E (20 mM tris-hydrochloride buffer (pH 8.5), 200 mM sodium chloride, 1 mM magnesium chloride, 10% glycerol), 5 ml of buffer F (20 mM tris-hydrochloride buffer (pH 8.5), 400 mM sodium chloride, 1 mM magnesium chloride, 10% glycerol) and 5 ml of buffer G (20 mM tris-hydrochloride buffer (pH 8.5), 800 mM sodium chloride, 1 mM magnesium chloride, 10% glycerol). Each of the eluted samples was concentrated about 20-fold using Centricon YM-10 (Amicon) to obtain an about 250 μl of a protein sample. When a portion of the protein sample was subjected to electrophoresis on 10% SDS-polyacrylamide gel, a band for the protein of interest was observed at a position corresponding to a molecular weight of about 144,000. This sample was used for subsequent confirmation of the activity. Comparison of the activity corresponding to 100 ml of the cultures for the sample expressed and purified as described above with that expressed and purified using Escherichia coli BL21-CodonPlus-RIL strain (Stratagene) as a host in Example 4-(2) indicated an about 2-fold greater yield.

(2) Measurement of dsRNA Degradation Activity

Using the protein sample prepared in Example 11-(1) above, a reaction mixture of a total volume of 10 μl was prepared by adding 1 μg of the dsRNA prepared in Example 2-(1), 1 μl of the protein sample prepared in 11-(1), 2 μl of 5× reaction buffer (100 mM tris-hydrochloride buffer (pH 8.5), 750 mM sodium chloride, 12.5 mM magnesium chloride) and nuclease-free water. After the reaction mixture was reacted at 37° C. for 18 hours, 5 μl of the reaction mixture was subjected to electrophoresis on 15% polyacrylamide gel and staining with ethidium bromide to observe the cleavage product. As a result, a degradation product of about 21 base pairs was observed, indicating its dsRNA degradation activity.

(3) Increase in Cleavage Activity by CspB Protein

Influence of a protein having an activity of binding to a nucleic acid (CspB) was examined using the protein sample prepared in Example 11-(1).

Specifically, like the procedure in Example 10-(3), a reaction mixture of a total volume of 10 μl was prepared by adding 1 μl of the protein sample prepared in Example 11-(1) (enzyme solution), 1 μl of the CspB solution prepared in Example 10-(2), 1 μg of the dsRNA as a substrate prepared in Example 2-(1), 2 μl of 5× reaction buffer (100 mM tris-hydrochloride buffer (pH 8.5), 750 mM sodium chloride, 12.5 mM magnesium chloride) and nuclease-free water. The final concentration of the CspB protein was 9.2 ng/μl. A similar procedure was carried out using a commercially available Dicer as a control. In case of the commercially available Dicer (GTS), a reaction mixture of a total volume of 10 μl was prepared by adding 2 μl of an enzyme solution, 1 μl of the CspB solution, 1 μg of the dsRNA as a substrate, 1 μl of 10 mM ATP solution, 0.5 μl of 50 mM magnesium chloride solution, 4 μl of the attached reaction buffer and nuclease-free water. Nuclease-free water was added in place of CspB for a control.

After the above-mentioned reaction mixtures were prepared and reacted at 37° C. for 18 hours, 5 μl of the reaction mixture was subjected to electrophoresis on 15% polyacrylamide gel and staining with ethidium bromide to observe cleavage products. The gel was subjected to image analysis using Total Lab ver. 1.11 (Nonlinear Dynamics) to analyze a dsRNA degradation product of about 21 nucleotides and an uncleaved dsRNA.

As a result, when the mutant protein containing the PAZ+RNase III domain of the present invention (hDi-ASI) was used, increase in the amount of degraded dsRNA due to the addition of CspB was observed, and almost no uncleaved substrate existed, indicating almost complete degradation. On the other hand, in case of the commercially available Dicer, increase in the amount of degraded dsRNA was observed although about half of the substrate remained uncleaved.

Thus, it was shown that the substrate could be completely degraded by adding CspB when the protein of the present invention was used.

### Example 12

### RNAi Effect of Cleavage Product Obtained Using hDi-ASI

The RNA interference effect of an siRNA prepared using the protein in Example 11-(1) was examined. A commercially available Dicer (GTS) was used as a control. dsRNA degradation products were prepared basically according to the method as described in Example 11-(3). In case of hDi-ASI, CspB prepared in Example 10-(2) was added at a final concentration of 9.2 ng/μl. Specifically, 10 μg of the dsRNA prepared in Example 2-(1) was cleaved at 37° C. for 18 hours using the enzyme as described in Example 11-(1) with the addition of CspB or the commercially available Dicer. The cleavage products were purified using Microcon-100 and Micropure-EZ (both from Takara Bio), and used for assessments in RNA interference as follows.

Briefly, an appropriate number (1.5×10⁵ cells) of 293 cells were seeded into a 24-well plate containing D-MEM medium (SIGMA) supplemented with 10% FBS and 1% penicillin/streptomycin 24 hours before transfer of an siRNA, and cultured overnight in a CO₂ incubator. The cells were cultured to about 95% confluence. 1 μl of Genejuice Transfection Reagent (Takara Bio) was added to 49 μl of a serum-free medium. The mixture was vigorously stirred and allowed to stand at room temperature for 5 minutes. 0.3 μg of pQBI25 (Takara Bio) was added thereto, and the mixture was gently mixed and allowed to stand at room temperature for 5 minutes.

In parallel, 3 μl of Ribojuice Transfection Reagent (Takara Bio) was added to 47 μl of a serum-free medium in another tube, and the mixture was vigorously stirred. After allowing to stand at room temperature for 5 minutes, 55.6 ng of the siRNA was added thereto, and the mixture was gently mixed and allowed to stand at room temperature for 5 minutes.

One of the two types of solutions prepared as described above was added dropwise to 250 μl of D-MEM medium supplemented with 10% FBS in each well, and the mixture was mixed gently so that the solution in the well became homogeneous. The vector (DNA) or sterile water was added alone to controls. The cells were cultured for 24 hours in a CO₂ incubator. The cells were subjected to flow cytometry using FACS Vantage (Becton-Dickinson) to determine the inhibitory effect of the transferred DNA/siRNA solution on rsGFP expression as compared with the control with the vector (DNA) alone. The results are shown in Table 6.

TABLE 6

| | Average |
|---|---|
| | fluorescence |
| Transferred sample | intensity |
| Control (no addition) | 3.52 |
| Control (vector alone) | 1120.08 |
| hDi-ASI + CspB | 93.09 |
| Commercially available Dicer | 278.59 |

A less average fluorescence value as compared with the control (vector alone) as shown in Table 6 represents more RNA interference. It was shown that the siRNA obtained using hDiR-ASI+CspB exhibited an RNAi effect like the one obtained using the commercially available Dicer and, therefore, it was effective for RNAi. Furthermore, it was confirmed that an siRNA could be efficiently produced, and a greater RNAi effect could be achieved as compared with a commercially available enzyme provided that the same amount of an siRNA was used.

Based on the above, it was confirmed that the protein prepared according to the method of the present invention is useful for preparation of an siRNA for RNA interference.

### INDUSTRIAL APPLICABILITY

The present invention provides a protein having an activity of degrading a dsRNA to produce a dsRNA of a specific length. Furthermore, a method for promoting degradation into a dsRNA of a specific length and/or a method for promoting synthesis of an RNA which is useful for RNA interference or the like is provided according to the method of the present invention. In addition, a composition and a kit which can be used to conveniently carry out the method for promoting degradation into a dsRNA of a specific length and/or the method for promoting synthesis of an RNA of the present invention are provided.

Sequence Listing Free Text

SEQ ID NO:3; A gene encoding human dicer mutant

SEQ ID NO:4; An amino acid sequence of human dicer mutant

SEQ ID NO:5; Synthetic primer 1 to amplify a gene encoding human dicer mutant

SEQ ID NO:6; Synthetic primer 2 to amplify a gene encoding human dicer mutant

SEQ ID NO:7; Synthetic primer 3 to amplify a gene encoding red-shifted green fluorescence protein

SEQ ID NO:8; Synthetic primer 4 to amplify a gene encoding red-shifted green fluorescence protein

SEQ ID NO:14; Synthetic primer 5 to amplify a gene encoding human dicer mutant

SEQ ID NO:15; Synthetic primer 6 to amplify a gene encoding human dicer mutant

SEQ ID NO:16; A gene encoding human dicer mutant

SEQ ID NO:17; An amino acid sequence of human dicer mutant

SEQ ID NO:18; An amino acid sequence of human dicer mutant

SEQ ID NO:19; A gene encoding human dicer mutant

SEQ ID NO:20; Synthetic primer rsGFP-F to amplify a gene encoding rsGFP

SEQ ID NO:21; Synthetic primer rsGFP-R to amplify a gene encoding rsGFP

SEQ ID NO:22; Synthetic primer Neo-F to amplify a gene encoding Neo

SEQ ID NO:23; Synthetic primer Neo-R to amplify a gene encoding Neo

SEQ ID NO:24; Synthetic primer dsl-1 to amplify a gene encoding luciferase

SEQ ID NO:25; Synthetic primer dsl-2 to amplify a gene encoding luciferase

SEQ ID NO:26; Synthetic primer E to amplify a gene encoding CspB

SEQ ID NO:27; Synthetic primer F to amplify a gene encoding CspB

1. A protein having an activity of degrading a dsRNA, which has an activity of acting on a dsRNA to produce a dsRNA of a specific length.

2. The protein according to claim 1, which has a functional domain of a Dicer.

3. The protein according to claim 2, wherein the functional domain of a Dicer consists of RNase IIIa, RNase IIIb and dsRNA-binding domains.

4. The protein according to claim 3, which further contains a PAZ domain.

5. The protein according to claim 1, wherein the dsRNA of specific length is a dsRNA of about 15 to 30 base pairs.

6. The protein according to claim 1, which is a protein consisting of the amino acid sequence of SEQ ID NO:4 or 17, or an amino acid sequence encoded by the nucleotide sequence of SEQ ID NO:3 or 16.

7. The protein according to claim 1, which is a protein consisting of an amino acid sequence in which one or plural amino acid(s) is(are) substituted, deleted, inserted or added in the amino acid sequence of SEQ ID NO:4 or 17.

8. A method for producing the protein having an activity of degrading a dsRNA defined by claim 1, the method comprising converting a codon into one that is suitable for expression in a host, or reinforcing a host to be used for a rare codon.

9. A method for producing the protein having an activity of degrading a dsRNA defined by claim 1, the method comprising expressing the protein using a cold-inducible vector.

10. A kit containing the protein having an activity of degrading a dsRNA defined by claim 1.

11. A method for degrading a dsRNA, the method comprising allowing a protein having an activity of degrading a dsRNA to act on a dsRNA in the presence of a protein having an activity of binding to a nucleic acid to produce a dsRNA of a specific length.

12. The method according to claim 11, wherein the protein having an activity of binding to a nucleic acid and the protein having an activity of degrading a dsRNA are a fusion protein.

13. The method according to claim 11, wherein the protein having an activity of binding to a nucleic acid is a protein having an activity of binding to an RNA.

14. The method according to claim 13, wherein the protein having an activity of binding to an RNA is a cold shock protein.

15. The method according to claim 14, wherein the cold shock protein is derived from a thermophilic bacterium or a thermostable bacterium.

16. The method according to claim 15, wherein the cold shock protein is cold shock protein B from Thermotoga maritima.

17. The method according to claim 11, wherein the dsRNA of specific length is a dsRNA of about 15 to 30 base pairs.

18. (canceled)

19. The method according to claim 11, wherein the protein having an activity of degrading a dsRNA is a native Dicer or a functional equivalent thereof.

20. A method for synthesizing an RNA, the method comprising conducting an RNA synthesis reaction using a protein having an activity of synthesizing an RNA in the presence of a protein having an activity of binding to a nucleic acid.

21. The method according to claim 20, wherein a fusion protein of the protein having an activity of binding to a nucleic acid and the protein having an activity of synthesizing an RNA is used.

22. The method according to claim 20, wherein the protein having an activity of binding to a nucleic acid is a cold shock protein.

23. The method according to claim 22, wherein the cold shock protein is derived from a thermophilic bacterium or a thermostable bacterium.

24. The method according to claim 23, wherein the cold shock protein is cold shock protein B from Thermotoga maritima.

25. The method according to claim 20, wherein the protein having an activity of synthesizing an RNA is a DNA-dependent RNA polymerase.

26. A composition used for the method defined by claim 11, the composition containing a protein having an activity of binding to a nucleic acid and a protein having an activity of degrading a dsRNA.

27. A kit used for the method defined by claim 11, the kit containing a protein having an activity of binding to a nucleic acid and a protein having an activity of degrading a dsRNA.

28. A composition used for the method defined by claim 20, the composition containing a protein having an activity of binding to a nucleic acid and a protein having an activity of synthesizing an RNA.

29. A kit used for the method defined by claim 20, the kit containing a protein having an activity of binding to a nucleic acid and a protein having an activity of synthesizing an RNA.


SEQUENCE LISTING (27 sequences)

SEQ ID NO: 1 - protein, 1924 aa, Homo sapiens [sequence omitted]

SEQ ID NO: 2 - DNA, 5772 bp, Homo sapiens
atgaaaagccctgctttgcaacccctcagcatggcaggcctgcagctcatgacccctgcttcctcaccaatgggtcctttctttggactgccatggcaacaagaagcaattcatgataacatttatacgccaagaaaatatcaggttgaactgcttgaagcagctctggatcataataccatcgtctgtttaaacactggctcagggaagacatttattgctagtactactctactaaagagctgtctctatctagatctaggggagacttcagctagaaatggaaaaaggacggtgttcttggtcaactctgcaaaccaggttgctcaacaagtgtcagctgtcagaactcattcagatctcaaggttggggaatactcaaacctagaagtaaatgcatcttggacaaaagagagatggaaccaagagtttactaagcaccaggttctcattatgacttgctatgtcgccttgaatgttttgaaaaatggttacttatcactgtcagacattaaccttttggtgtttgatgagtgtcatcttgcaatcctagaccacccctatcgagaatttatgaagctctgtgaaatttgtccatcatgtcctcgcattttgggactaactgcttccattttaaatgggaaatgggatccagaggatttggaagaaaagtttcagaaactagagaaaattcttaagagtaatgctgaaactgcaactgacctggtggtcttagacaggtatacttctcagccatgtgagattgtggtggattgtggaccatttactgacagaagtgggctttatgaaagactgctgatggaattagaagaagcacttaattttatcaatgattgtaatatatctgtacattcaaaagaaagagattctactttaatttcgaaacagatactatcagactgtcgtgccgtattggtagttctgggaccctggtgtgcagataaagtagctggaatgatggtaagagaactacagaaatacatcaaacatgagcaagaggagctgcacaggaaatttttattgtttacagacactttcctaaggaaaatacatgcactatgtgaagagcacttctcacctgcctcacttgacctgaaatttgtaactcctaaagtaatcaaactgctcgaaatcttacgcaaatataaaccatatgagcgacacagttttgaaagcgttgagtggtataataatagaaatcaggataattatgtgtcatggagtgattctgaggatgatgatgaggatgaagaaattgaagaaaaagagaagccagagacaaattttccttctccttttaccaacattttgtgcggaattatttttgtggaaagaagatacacagcagttgtcttaaacagattgataaaggaagctggcaaacaagatccagagctggcttatatcagtagcaatttcataactggacatggcattgggaagaatcagcctcgcaacaacacgatggaagcagaattcagaaaacaggaagaggtacttaggaaatttcgagcacatgagaccaacctgcttattgcaacaagtattgtagaagagggtgttgatataccaaaatgcaacttggtggttcgttttgatttgcccacagaatatcgatcctatgttcaatctaaaggaagagcaagggcacccatctctaattatataatgttagcggatacagacaaaataaaaagttttgaagaagaccttaaaacctacaaagctattgaaaagatcttgagaaacaagtgttccaagtcggttgatactggtgagactgacattgatcctgtcatggatgatgatcacgttttcccaccatatgtgttgaggcctgacgatggtggtccacgagtcacaatcaacacggccattggacacatcaatagatactgtgctagattaccaagtgatccgtttactcatctagctcctaaatgcagaacccgagagttgcctgatggtacattttattcaactctttatctgccaattaactcacctcttcgagcctccattgttggtccaccaatgagctgtgtacgattggctgaaagagttgtcgctctcatttgctgtgagaaactgcacaaaattggcgaactggatgaccatttgatgccagttgggaaagagactgttaaatatgaagaggagcttgatttgcatgatgaagaagagaccagtgttccaggaagaccaggttccacgaaacgaaggcagtgctacccaaaagcaattccagagtgtttgagggatagttatcccagacctgatcagccctgttacctgtatgtgataggaatggttttaactacacctttacctgatgaactcaactttagaaggcggaagctctatcctcctgaagataccacaagatgctttggaatactgacggccaaacccatacctcagattccacactttcctgtgtacacacgctctggagaggttaccatatccattgagttgaagaagtctggtttcatgttgtctctacaaatgcttgagttgattacaagacttcaccagtatatattctcacatattcttcggcttgaaaaacctgcactagaatttaaacctacagacgctgattcagcatactgtgttctacctcttaatgttgttaatgactccagcactttggatattgactttaaattcatggaagatattgagaagtctgaagctcgcataggcattcccagtacaaagtatacaaaagaaacaccctttgtttttaaattagaagattaccaagatgccgttatcattccaagatatcgcaattttgatcagcctcatcgattttatgtagctgatgtgtacactgatcttaccccactcagtaaatttccttcccctgagtatgaaacttttgcagaatattataaaacaaagtacaaccttgacctaaccaatctcaaccagccactgctggatgtggaccacacatcttcaagacttaatcttttgacacctcgacatttgaatcagaaggggaaagcgcttcctttaagcagtgctgagaagaggaaagccaaatgggaaagtctgcagaataaacagatactggttccagaactctgtgctatacatccaattccagcatcactgtggagaaaagctgtttgtctccccagcatactttatcgccttcactgccttttgactgcagaggagctaagagcccagactgccagcgatgctggcgtgggagtcagatcacttcctgcggattttagataccctaacttagacttcgggtggaaaaaatctattgacagcaaatctttcatctcaatttctaactcctcttcagctgaaaatgataattactgtaagcacagcacaattgtccctgaaaatgctgcacatcaaggtgctaatagaacctcctctctagaaaatcatgaccaaatgtctgtgaactgcagaacgttgctcagcgagtcccctggtaagctccacgttgaagtttcagcagatcttacagcaattaatggtctttcttacaatcaaaatctcgccaatggcagttatgatttagctaacagagacttttgccaaggaaatcagctaaattactacaagcaggaaatacccgtgcaaccaactacctcatattccattcagaatttatacagttacgagaaccagccccagcccagcgatgaatgtactctcctgagtaataaataccttgatggaaatgctaacaaatctacctcagatggaagtcctgtgatggccgtaatgcctggtacgacagacactattcaagtgctcaagggcaggatggattctgagcagagcccttctattgggtactcctcaaggactcttggccccaatcctggacttattcttcaggctttgactctgtcaaacgctagtgatggatttaacctggagcggcttgaaatgcttggcgactcctttttaaagcatgccatcaccacatatctattttgcacttaccctgatgcgcatgagggccgcctttcatatatgagaagcaaaaaggtcagcaactgtaatctgtatcgccttggaaaaaagaagggactacccagccgcatggtggtgtcaatatttgatccccctgtgaattggcttcctcctggttatgtagtaaatcaagacaaaagcaacacagataaatgggaaaaagatgaaatgacaaaagactgcatgctggcgaatggcaaactggatgaggattacgaggaggaggatgaggaggaggagagcctgatgtggagggctccgaaggaagaggctgactatgaagatgatttcctggagtatgatcaggaacatatcagatttatagataatatgttaatggggtcaggagcttttgtaaagaaaatctctctttctcctttttcaaccactgattctgcatatgaatggaaaatgcccaaaaaatcctccttaggtagtatgccattttcatcagattttgaggattttgactacagctcttgggatgcaatgtgctatctggatcctagcaaagctgttgaagaagatgactttgtggtggggttctggaatccatcagaagaaaactgtggtgttgacacgggaaagcagtccatttcttacgacttgcacactgagcagtgtattgctgacaaaagcatagcggactgtgtggaagccctgctgggctgctatttaaccagctgtggggagagggctgctcagcttttcctctgttcactggggctgaaggtgctcccggtaattaaaaggactgatcgggaaaaggccctgtgccctactcgggagaatttcaacagccaacaaaagaacctttcagtgagctgtgctgctgcttctgtggccagttcacgctcttctgtattgaaagactcggaatatggttgtttgaagattccaccaagatgtatgtttgatcatccagatgcagataaaacactgaatcaccttatatcggggtttgaaaattttgaaaagaaaatcaactacagattcaagaataaggcttaccttctccaggcttttacacatgcctcctaccactacaatactatcactgattgttaccagcgcttagaattcctgggagatgcgattttggactacctcataaccaagcacctttatgaagacccgcggcagcactccccgggggtcctgacagacctgcggtctgccctggtcaacaacaccatctttgcatcgctggctgtaaagtacgactaccacaagtacttcaaagctgtctctcctgagctcttccatgtcattgatgactttgtgcagtttcagcttgagaagaatgaaatgcaaggaatggattctgagcttaggagatctgaggaggatgaagagaaagaagaggatattgaagttccaaaggccatgggggatatttttgagtcgcttgctggtgccatttacatggatagtgggatgtcactggagacagtctggcaggtgtactatcccatgatgcggccactaatagaaaagttttctgcaaatgtaccccgttcccctgtgcgagaattgcttgaaatggaaccagaaactgccaaatttagcccggctgagagaacttacgacgggaaggtcagagtcactgtggaagtagtaggaaaggggaaatttaaaggtgttggtcgaagttacaggattgccaaatctgcagcagcaagaagagccctccgaagcctcaaagctaatcaacctcaggttcccaatagc

SEQ ID NO: 3 - DNA, 1962 bp, Artificial sequence
  A gene encoding human dicer mutant
caagtgctcaagggcaggatggattctgagcagagcccttctattgggtactcctcaaggactcttggccccaatcctggacttattcttcaggctttgactctgtcaaacgctagtgatggatttaacctggagcggcttgaaatgcttggcgactcctttttaaagcatgccatcaccacatatctattttgcacttaccctgatgcgcatgagggccgcctttcatatatgagaagcaaaaaggtcagcaactgtaatctgtatcgccttggaaaaaagaagggactacccagccgcatggtggtgtcaatatttgatccccctgtgaattggcttcctcctggttatgtagtaaatcaagacaaaagcaacacagataaatgggaaaaagatgaaatgacaaaagactgcatgctggcgaatggcaaactggatgaggattacgaggaggaggatgaggaggaggagagcctgatgtggagggctccgaaggaagaggctgactatgaagatgatttcctggagtatgatcaggaacatatcagatttatagataatatgttaatggggtcaggagcttttgtaaagaaaatctctctttctcctttttcaaccactgattctgcatatgaatggaaaatgcccaaaaaatcctccttaggtagtatgccattttcatcagattttgaggattttgactacagctcttgggatgcaatgtgctatctggatcctagcaaagctgttgaagaagatgactttgtggtggggttctggaatccatcagaagaaaactgtggtgttgacacgggaaagcagtccatttcttacgacttgcacactgagcagtgtattgctgacaaaagcatagcggactgtgtggaagccctgctgggctgctatttaaccagctgtggggagagggctgctcagcttttcctctgttcactggggctgaaggtgctcccggtaattaaaaggactgatcgggaaaaggccctgtgccctactcgggagaatttcaacagccaacaaaagaacctttcagtgagctgtgctgctgcttctgtggccagttcacgctcttctgtattgaaagactcggaatatggttgtttgaagattccaccaagatgtatgtttgatcatccagatgcagataaaacactgaatcaccttatatcggggtttgaaaattttgaaaagaaaatcaactacagattcaagaataaggcttaccttctccaggcttttacacatgcctcctaccactacaatactatcactgattgttaccagcgcttagaattcctgggagatgcgattttggactacctcataaccaagcacctttatgaagacccgcggcagcactccccgggggtcctgacagacctgcggtctgccctggtcaacaacaccatctttgcatcgctggctgtaaagtacgactaccacaagtacttcaaagctgtctctcctgagctcttccatgtcattgatgactttgtgcagtttcagcttgagaagaatgaaatgcaaggaatggattctgagcttaggagatctgaggaggatgaagagaaagaagaggatattgaagttccaaaggccatgggggatatttttgagtcgcttgctggtgccatttacatggatagtgggatgtcactggagacagtctggcaggtgtactatcccatgatgcggccactaatagaaaagttttctgcaaatgtaccccgttcccctgtgcgagaattgcttgaaatggaaccagaaactgccaaatttagcccggctgagagaacttacgacgggaaggtcagagtcactgtggaagtagtaggaaaggggaaatttaaaggtgttggtcgaagttacaggattgccaaatctgcagcagcaagaagagccctccgaagcctcaaagctaatcaacctcaggttcccaatagc

SEQ ID NO: 4 - protein, 654 aa, Artificial sequence
  An amino acid sequence of human dicer mutant.
QVLKGRMDSEQSPSIGYSSRTLGPNPGLILQALTLSNASDGFNLERLEMLGDSFLKHAITTYLFCTYPDAHEGRLSYMRSKKVSNCNLYRLGKKKGLPSRMVVSIFDPPVNWLPPGYVVNQDKSNTDKWEKDEMTKDCMLANGKLDEDYEEEDEEEESLMWRAPKEEADYEDDFLEYDQEHIRFIDNMLMGSGAFVKKISLSPFSTTDSAYEWKMPKKSSLGSMPFSSDFEDFDYSSWDAMCYLDPSKAVEEDDFVVGFWNPSEENCGVDTGKQSISYDLHTEQCIADKSIADCVEALLGCYLTSCGERAAQLFLCSLGLKVLPVIKRTDREKALCPTRENFNSQQKNLSVSCAAASVASSRSSVLKDSEYGCLKIPPRCMFDHPDADKTLNHLISGFENFEKKINYRFKNKAYLLQAFTHASYHYNTITDCYQRLEFLGDAILDYLITKHLYEDPRQHSPGVLTDLRSALVNNTIFASLAVKYDYHKYFKAVSPELFHVIDDFVQFQLEKNEMQGMDSELRRSEEDEEKEEDIEVPKAMGDIFESLAGAIYMDSGMSLETVWQVYYPMMRPLIEKFSANVPRSPVRELLEMEPETAKFSPAERTYDGKVRVTVEVVGKGKFKGVGRSYRIAKSAAARRALRSLKANQPQVPNS

SEQ ID NO: 5 - DNA, 36 bp, Artificial sequence
  Synthetic primer 1 to amplify a gene encoding human dicer
tcgagctcggtaccccaagtgctcaagggcaggatg

SEQ ID NO: 6 - DNA, 36 bp, Artificial sequence
  Synthetic primer 2 to amplify a gene encoding human dicer
tatctagaaagcttttagctattgggaacctgaggt

SEQ ID NO: 7 - DNA, 42 bp, Artificial sequence
  Synthetic primer 3 to amplify a gene encoding red-shifted green fluorescence protein
gggtaatacgactcactatagggagaatggctagcaaaggag

SEQ ID NO: 8 - DNA, 42 bp, Artificial sequence
  Synthetic primer 4 to amplify a gene encoding red-shifted green fluorescence protein
gggtaatacgactcactatagggagatcagttgtacagttca

SEQ ID NO: 9 - protein, 66 aa, Thermotoga maritima
MRGKVKWFDSKKGYGFITKDEGGDVFVHWSAIEMEGFKTLKEGQVVEFEIQEGKKGPQAAHVKVVE

SEQ ID NO: 10 - DNA, 198 bp, Thermotoga maritima
atgagaggaaaggttaagtggttcgattccaagaagggctacggattcatcacaaaggacgaaggaggagacgtgttcgtacactggtcagccatcgaaatggaaggtttcaaaactctgaaggaaggccaggtcgtcgagttcgagattcaggaaggcaagaaaggtccacaggcagcgcacgtgaaagtagttgag

SEQ ID NO: 11 - DNA, 720 bp, Artificial sequence
  A gene encoding red-shifted green fluorescence protein.
atggctagcaaaggagaagaactcttcactggagttgtcccaattcttgttgaattagatggtgatgttaacggccacaagttctctgtcagtggagagggtgaaggtgatgcaacatacggaaaacttaccctgaagttcatctgcactactggcaaactgcctgttccatggccaacactagtcactactctgtgctatggtgttcaatgcttttcaagatacccggatcatatgaaacggcatgactttttcaagagtgccatgcccgaaggttatgtacaggaaaggaccatcttcttcaaagatgacggcaactacaagacacgtgctgaagtcaagtttgaaggtgatacccttgttaatagaatcgagttaaaaggtattgacttcaaggaagatggaaacattctgggacacaaattggaatacaactataactcacacaatgtatacatcatggcagacaaacaaaagaatggaatcaaagtgaacttcaagacccgccacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactgtacaactga

SEQ ID NO: 12 - protein, 675 aa, Artificial sequence
  An amino acid sequence of human dicer mutant
MNHKVHHHHHHIEGRNSSSVPQVLKGRMDSEQSPSIGYSSRTLGPNPGLILQALTLSNASDGFNLERLEMLGDSFLKHAITTYLFCTYPDAHEGRLSYMRSKKVSNCNLYRLGKKKGLPSRMVVSIFDPPVNWLPPGYVVNQDKSNTDKWEKDEMTKDCMLANGKLDEDYEEEDEEEESLMWRAPKEEADYEDDFLEYDQEHIRFIDNMLMGSGAFVKKISLSPFSTTDSAYEWKMPKKSSLGSMPFSSDFEDFDYSSWDAMCYLDPSKAVEEDDFVVGFWNPSEENCGVDTGKQSISYDLHTEQCIADKSIADCVEALLGCYLTSCGERAAQLFLCSLGLKVLPVIKRTDREKALCPTRENFNSQQKNLSVSCAAASVASSRSSVLKDSEYGCLKIPPRCMFDHPDADKTLNHLISGFENFEKKINYRFKNKAYLLQAFTHASYHYNTITDCYQRLEFLGDAILDYLITKHLYEDPRQHSPGVLTDLRSALVNNTIFASLAVKYDYHKYFKAVSPELFHVIDDFVQFQLEKNEMQGMDSELRRSEEDEEKEEDIEVPKAMGDIFESLAGAIYMDSGMSLETVWQVYYPMMRPLIEKFSANVPRSPVRELLEMEPETAKFSPAERTYDGKVRVTVEVVGKGKFKGVGRSYRIAKSAAARRALRSLKANQPQVPNS

SEQ ID NO: 13 - DNA, 2025 bp, Artificial sequence
  A gene encoding human dicer mutant
atgaatcacaaagtgcatcatcatcatcatcatatcgaaggtaggaattcgagctcggtaccccaagtgctcaagggcaggatggattctgagcagagcccttctattgggtactcctcaaggactcttggccccaatcctggacttattcttcaggctttgactctgtcaaacgctagtgatggatttaacctggagcggcttgaaatgcttggcgactcctttttaaagcatgccatcaccacatatctattttgcacttaccctgatgcgcatgagggccgcctttcatatatgagaagcaaaaaggtcagcaactgtaatctgtatcgccttggaaaaaagaagggactacccagccgcatggtggtgtcaatatttgatccccctgtgaattggcttcctcctggttatgtagtaaatcaagacaaaagcaacacagataaatgggaaaaagatgaaatgacaaaagactgcatgctggcgaatggcaaactggatgaggattacgaggaggaggatgaggaggaggagagcctgatgtggagggctccgaaggaagaggctgactatgaagatgatttcctggagtatgatcaggaacatatcagatttatagataatatgttaatggggtcaggagcttttgtaaagaaaatctctctttctcctttttcaaccactgattctgcatatgaatggaaaatgcccaaaaaatcctccttaggtagtatgccattttcatcagattttgaggattttgactacagctcttgggatgcaatgtgctatctggatcctagcaaagctgttgaagaagatgactttgtggtggggttctggaatccatcagaagaaaactgtggtgttgacacgggaaagcagtccatttcttacgacttgcacactgagcagtgtattgctgacaaaagcatagcggactgtgtggaagccctgctgggctgctatttaaccagctgtggggagagggctgctcagcttttcctctgttcactggggctgaaggtgctcccggtaattaaaaggactgatcgggaaaaggccctgtgccctactcgggagaatttcaacagccaacaaaagaacctttcagtgagctgtgctgctgcttctgtggccagttcacgctcttctgtattgaaagactcggaatatggttgtttgaagattccaccaagatgtatgtttgatcatccagatgcagataaaacactgaatcaccttatatcggggtttgaaaattttgaaaagaaaatcaactacagattcaagaataaggcttaccttctccaggcttttacacatgcctcctaccactacaatactatcactgattgttaccagcgcttagaattcctgggagatgcgattttggactacctcataaccaagcacctttatgaagacccgcggcagcactccccgggggtcctgacagacctgcggtctgccctggtcaacaacaccatctttgcatcgctggctgtaaagtacgactaccacaagtacttcaaagctgtctctcctgagctcttccatgtcattgatgactttgtgcagtttcagcttgagaagaatgaaatgcaaggaatggattctgagcttaggagatctgaggaggatgaagagaaagaagaggatattgaagttccaaaggccatgggggatatttttgagtcgcttgctggtgccatttacatggatagtgggatgtcactggagacagtctggcaggtgtactatcccatgatgcggccactaatagaaaagttttctgcaaatgtaccccgttcccctgtgcgagaattgcttgaaatggaaccagaaactgccaaatttagcccggctgagagaacttacgacgggaaggtcagagtcactgtggaagtagtaggaaaggggaaatttaaaggtgttggtcgaagttacaggattgccaaatctgcagcagcaagaagagccctccgaagcctcaaagctaatcaacctcaggttcccaatagc

SEQ ID NO: 14 - DNA, 36 bp, Artificial
  Synthetic primer 5 to amplify a gene encoding human dicer mutant
tcgagctcggtacccgcctccattgttggtccacca

SEQ ID NO: 15 - DNA, 36 bp, Artificial; identical to SEQ ID NO: 6
  Synthetic primer 6 to amplify a gene encoding human dicer mutant

SEQ ID NO: 16 - DNA, 3741 bp, Artificial
  A gene encoding human dicer mutant
gcctccattgttggtccaccaatgagctgtgtacgattggctgaaagagttgtcgctctcatttgctgtgagaaactgcacaaaattggcgaactggatgaccatttgatgccagttgggaaagagactgttaaatatgaagaggagcttgatttgcatgatgaagaagagaccagtgttccaggaagaccaggttccacgaaacgaaggcagtgctacccaaaagcaattccagagtgtttgagggatagttatcccagacctgatcagccctgttacctgtatgtgataggaatggttttaactacacctttacctgatgaactcaactttagaaggcggaagctctatcctcctgaagataccacaagatgctttggaatactgacggccaaacccatacctcagattccacactttcctgtgtacacacgctctggagaggttaccatatccattgagttgaagaagtctggtttcatgttgtctctacaaatgcttgagttgattacaagacttcaccagtatatattctcacatattcttcggcttgaaaaacctgcactagaatttaaacctacagacgctgattcagcatactgtgttctacctcttaatgttgttaatgactccagcactttggatattgactttaaattcatggaagatattgagaagtctgaagctcgcataggcattcccagtacaaagtatacaaaagaaacaccctttgtttttaaattagaagattaccaagatgccgttatcattccaagatatcgcaattttgatcagcctcatcgattttatgtagctgatgtgtacactgatcttaccccactcagtaaatttccttcccctgagtatgaaacttttgcagaatattataaaacaaagtacaaccttgacctaaccaatctcaaccagccactgctggatgtggaccacacatcttcaagacttaatcttttgacacctcgacatttgaatcagaaggggaaagcgcttcctttaagcagtgctgagaagaggaaagccaaatgggaaagtctgcagaataaacagatactggttccagaactctgtgctatacatccaattccagcatcactgtggagaaaagctgtttgtctccccagcatactttatcgccttcactgccttttgactgcagaggagctaagagcccagactgccagcgatgctggcgtgggagtcagatcacttcctgcggattttagataccctaacttagacttcgggtggaaaaaatctattgacagcaaatctttcatctcaatttctaactcctcttcagctgaaaatgataattactgtaagcacagcacaattgtccctgaaaatgctgcacatcaaggtgctaatagaacctcctctctagaaaatcatgaccaaatgtctgtgaactgcagaacgttgctcagcgagtcccctggtaagctccacgttgaagtttcagcagatcttacagcaattaatggtctttcttacaatcaaaatctcgccaatggcagttatgatttagctaacagagacttttgccaaggaaatcagctaaattactacaagcaggaaatacccgtgcaaccaactacctcatattccattcagaatttatacagttacgagaaccagccccagcccagcgatgaatgtactctcctgagtaataaataccttgatggaaatgctaacaaatctacctcagatggaagtcctgtgatggccgtaatgcctggtacgacagacactattcaagtgctcaagggcaggatggattctgagcagagcccttctattgggtactcctcaaggactcttggccccaatcctggacttattcttcaggctttgactctgtcaaacgctagtgatggatttaacctggagcggcttgaaatgcttggcgactcctttttaaagcatgccatcaccacatatctattttgcacttaccctgatgcgcatgagggccgcctttcatatatgagaagcaaaaaggtcagcaactgtaatctgtatcgccttggaaaaaagaagggactacccagccgcatggtggtgtcaatatttgatccccctgtgaattggcttcctcctggttatgtagtaaatcaagacaaaagcaacacagataaatgggaaaaagatgaaatgacaaaagactgcatgctggcgaatggcaaactggatgaggattacgaggaggaggatgaggaggaggagagcctgatgtggagggctccgaaggaagaggctgactatgaagatgatttcctggagtatgatcaggaacatatcagatttatagataatatgttaatggggtcaggagcttttgtaaagaaaatctctctttctcctttttcaaccactgattctgcatatgaatggaaaatgcccaaaaaatcctccttaggtagtatgccattttcatcagattttgaggattttgactacagctcttgggatgcaatgtgctatctggatcctagcaaagctgttgaagaagatgactttgtggtggggttctggaatccatcagaagaaaactgtggtgttgacacgggaaagcagtccatttcttacgacttgcacactgagcagtgtattgctgacaaaagcatagcggactgtgtggaagccctgctgggctgctatttaaccagctgtggggagagggctgctcagcttttcctctgttcactggggctgaaggtgctcccggtaattaaaaggactgatcgggaaaaggccctgtgccctactcgggagaatttcaacagccaacaaaagaacctttcagtgagctgtgctgctgcttctgtggccagttcacgctcttctgtattgaaagactcggaatatggttgtttgaagattccaccaagatgtatgtttgatcatccagatgcagataaaacactgaatcaccttatatcggggtttgaaaattttgaaaagaaaatcaactacagattcaagaataaggcttaccttctccaggcttttacacatgcctcctaccactacaatactatcactgattgttaccagcgcttagaattcctgggagatgcgattttggactacctcataaccaagcacctttatgaagacccgcggcagcactccccgggggtcctgacagacctgcggtctgccctggtcaacaacaccatctttgcatcgctggctgtaaagtacgactaccacaagtacttcaaagctgtctctcctgagctcttccatgtcattgatgactttgtgcagtttcagcttgagaagaatgaaatgcaaggaatggattctgagcttaggagatctgaggaggatgaagagaaagaagaggatattgaagttccaaaggccatgggggatatttttgagtcgcttgctggtgccatttacatggatagtgggatgtcactggagacagtctggcaggtgtactatcccatgatgcggccactaatagaaaagttttctgcaaatgtaccccgttcccctgtgcgagaattgcttgaaatggaaccagaaactgccaaatttagcccggctgagagaacttacgacgggaaggtcagagtcactgtggaagtagtaggaaaggggaaatttaaaggtgttggtcgaagttacaggattgccaaatctgcagcagcaagaagagccctccgaagcctcaaagctaatcaacctcaggttcccaatagctaa

SEQ ID NO: 17 - protein, 1246 aa, Artificial
  An amino acid sequence of human dicer mutant
ASIVGPPMSCVRLAERVVALICCEKLHKIGELDDHLMPVGKETVKYEEELDLHDEEETSVPGRPGSTKRRQCYPKAIPECLRDSYPRPDQPCYLYVIGMVLTTPLPDELNFRRRKLYPPEDTTRCFGILTAKPIPQIPHFPVYTRSGEVTISIELKKSGFMLSLQMLELITRLHQYIFSHILRLEKPALEFKPTDADSAYCVLPLNVVNDSSTLDIDFKFMEDIEKSEARIGIPSTKYTKETPFVFKLEDYQDAVIIPRYRNFDQPHRFYVADVYTDLTPLSKFPSPEYETFAEYYKTKYNLDLTNLNQPLLDVDHTSSRLNLLTPRHLNQKGKALPLSSAEKRKAKWESLQNKQILVPELCAIHPIPASLWRKAVCLPSILYRLHCLLTAEELRAQTASDAGVGVRSLPADFRYPNLDFGWKKSIDSKSFISISNSSSAENDNYCKHSTIVPENAAHQGANRTSSLENHDQMSVNCRTLLSESPGKLHVEVSADLTAINGLSYNQNLANGSYDLANRDFCQGNQLNYYKQEIPVQPTTSYSIQNLYSYENQPQPSDECTLLSNKYLDGNANKSTSDGSPVMAVMPGTTDTIQVLKGRMDSEQSPSIGYSSRTLGPNPGLILQALTLSNASDGFNLERLEMLGDSFLKHAITTYLFCTYPDAHEGRLSYMRSKKVSNCNLYRLGKKKGLPSRMVVSIFDPPVNWLPPGYVVNQDKSNTDKWEKDEMTKDCMLANGKLDEDYEEEDEEEESLMWRAPKEEADYEDDFLEYDQEHIRFIDNMLMGSGAFVKKISLSPFSTTDSAYEWKMPKKSSLGSMPFSSDFEDFDYSSWDAMCYLDPSKAVEEDDFVVGFWNPSEENCGVDTGKQSISYDLHTEQCIADKSIADCVEALLGCYLTSCGERAAQLFLCSLGLKVLPVIKRTDREKALCPTRENFNSQQKNLSVSCAAASVASSRSSVLKDSEYGCLKIPPRCMFDHPDADKTLNHLISGFENFEKKINYRFKNKAYLLQAFTHASYHYNTITDCYQRLEFLGDAILDYLITKHLYEDPRQHSPGVLTDLRSALVNNTIFASLAVKYDYHKYFKAVSPELFHVIDDFVQFQLEKNEMQGMDSELRRSEEDEEKEEDIEVPKAMGDIFESLAGAIYMDSGMSLETVWQVYYPMMRPLIEKFSANVPRSPVRELLEMEPETAKFSPAERTYDGKVRVTVEVVGKGKFKGVGRSYRIAKSAAARRALRSLKANQPQVPNS

SEQ ID NO: 18 - protein, 1267 aa, Artificial
  An amino acid sequence of human dicer mutant
MNHKVHHHHHHIEGRNSSSVPASIVGPPMSCVRLAERVVALICCEKLHKIGELDDHLMPVGKETVKYEEELDLHDEEETSVPGRPGSTKRRQCYPKAIPECLRDSYPRPDQPCYLYVIGMVLTTPLPDELNFRRRKLYPPEDTTRCFGILTAKPIPQIPHFPVYTRSGEVTISIELKKSGFMLSLQMLELITRLHQYIFSHILRLEKPALEFKPTDADSAYCVLPLNVVNDSSTLDIDFKFMEDIEKSEARIGIPSTKYTKETPFVFKLEDYQDAVIIPRYRNFDQPHRFYVADVYTDLTPLSKFPSPEYETFAEYYKTKYNLDLTNLNQPLLDVDHTSSRLNLLTPRHLNQKGKALPLSSAEKRKAKWESLQNKQILVPELCAIHPIPASLWRKAVCLPSILYRLHCLLTAEELRAQTASDAGVGVRSLPADFRYPNLDFGWKKSIDSKSFISISNSSSAENDNYCKHSTIVPENAAHQGANRTSSLENHDQMSVNCRTLLSESPGKLHVEVSADLTAINGLSYNQNLANGSYDLANRDFCQGNQLNYYKQEIPVQPTTSYSIQNLYSYENQPQPSDECTLLSNKYLDGNANKSTSDGSPVMAVMPGTTDTIQVLKGRMDSEQSPSIGYSSRTLGPNPGLILQALTLSNASDGFNLERLEMLGDSFLKHAITTYLFCTYPDAHEGRLSYMRSKKVSNCNLYRLGKKKGLPSRMVVSIFDPPVNWLPPGYVVNQDKSNTDKWEKDEMTKDCMLANGKLDEDYEEEDEEEESLMWRAPKEEADYEDDFLEYDQEHIRFIDNMLMGSGAFVKKISLSPFSTTDSAYEWKMPKKSSLGSMPFSSDFEDFDYSSWDAMCYLDPSKAVEEDDFVVGFWNPSEENCGVDTGKQSISYDLHTEQCIADKSIADCVEALLGCYLTSCGERAAQLFLCSLGLKVLPVIKRTDREKALCPTRENFNSQQKNLSVSCAAASVASSRSSVLKDSEYGCLKIPPRCMFDHPDADKTLNHLISGFENFEKKINYRFKNKAYLLQAFTHASYHYNTITDCYQRLEFLGDAILDYLITKHLYEDPRQHSPGVLTDLRSALVNNTIFASLAVKYDYHKYFKAVSPELFHVIDDFVQFQLEKNEMQGMDSELRRSEEDEEKEEDIEVPKAMGDIFESLAGAIYMDSGMSLETVWQVYYPMMRPLIEKFSANVPRSPVRELLEMEPETAKFSPAERTYDGKVRVTVEVVGKGKFKGVGRSYRIAKSAAARRALRSLKANQPQVPNS

SEQ ID NO: 19 - DNA, 3804 bp, Artificial
  A gene encoding human dicer mutant
atgaatcacaaagtgcatcatcatcatcatcatatcgaaggtaggaattcgagctcggtacccgcctccattgttggtccaccaatgagctgtgtacgattggctgaaagagttgtcgctctcatttgctgtgagaaactgcacaaaattggcgaactggatgaccatttgatgccagttgggaaagagactgttaaatatgaagaggagcttgatttgcatgatgaagaagagaccagtgttccaggaagaccaggttccacgaaacgaaggcagtgctacccaaaagcaattccagagtgtttgagggatagttatcccagacctgatcagccctgttacctgtatgtgataggaatggttttaactacacctttacctgatgaactcaactttagaaggcggaagctctatcctcctgaagataccacaagatgctttggaatactgacggccaaacccatacctcagattccacactttcctgtgtacacacgctctggagaggttaccatatccattgagttgaagaagtctggtttcatgttgtctctacaaatgcttgagttgattacaagacttcaccagtatatattctcacatattcttcggcttgaaaaacctgcactagaatttaaacctacagacgctgattcagcatactgtgttctacctcttaatgttgttaatgactccagcactttggatattgactttaaattcatggaagatattgagaagtctgaagctcgcataggcattcccagtacaaagtatacaaaagaaacaccctttgtttttaaattagaagattaccaagatgccgttatcattccaagatatcgcaattttgatcagcctcatcgattttatgtagctgatgtgtacactgatcttaccccactcagtaaatttccttcccctgagtatgaaacttttgcagaatattataaaacaaagtacaaccttgacctaaccaatctcaaccagccactgctggatgtggaccacacatcttcaagacttaatcttttgacacctcgacatttgaatcagaaggggaaagcgcttcctttaagcagtgctgagaagaggaaagccaaatgggaaagtctgcagaataaacagatactggttccagaactctgtgctatacatccaattccagcatcactgtggagaaaagctgtttgtctccccagcatactttatcgccttcactgccttttgactgcagaggagctaagagcccagactgccagcgatgctggcgtgggagtcagatcacttcctgcggattttagataccctaacttagacttcgggtggaaaaaatctattgacagcaaatctttcatctcaatttctaactcctcttcagctgaaaatgataattactgtaagcacagcacaattgtccctgaaaatgctgcacatcaaggtgctaatagaacctcctctctagaaaatcatgaccaaatgtctgtgaactgcagaacgttgctcagcgagtcccctggtaagctccacgttgaagtttcagcagatcttacagcaattaatggtctttcttacaatcaaaatctcgccaatggcagttatgatttagctaacagagacttttgccaaggaaatcagctaaattactacaagcaggaaatacccgtgcaaccaactacctcatattccattcagaatttatacagttacgagaaccagccccagcccagcgatgaatgtactctcctgagtaataaataccttgatggaaatgctaacaaatctacctcagatggaagtcctgtgatggccgtaatgcctggtacgacagacactattcaagtgctcaagggcaggatggattctgagcagagcccttctattgggtactcctcaaggactcttggccccaatcctggacttattcttcaggctttgactctgtcaaacgctagtgatggatttaacctggagcggcttgaaatgcttggcgactcctttttaaagcatgccatcaccacatatctattttgcacttaccctgatgcgcatgagggccgcctttcatatatgagaagcaaaaaggtcagcaactgtaatctgtatcgccttggaaaaaagaagggactacccagccgcatggtggtgtcaatatttgatccccctgtgaattggcttcctcctggttatgtagtaaatcaagacaaaagcaacacagataaatgggaaaaagatgaaatgacaaaagactgcatgctggcgaatggcaaactggatgaggattacgaggaggaggatgaggaggaggagagcctgatgtggagggctccgaaggaagaggctgactatgaagatgatttcctggagtatgatcaggaacatatcagatttatagataatatgttaatggggtcaggagcttttgtaaagaaaatctctctttctcctttttcaaccactgattctgcatatgaatggaaaatgcccaaaaaatcctccttaggtagtatgccattttcatcagattttgaggattttgactacagctcttgggatgcaatgtgctatctggatcctagcaaagctgttgaagaagatgactttgtggtggggttctggaatccatcagaagaaaactgtggtgttgacacgggaaagcagtccatttcttacgacttgcacactgagcagtgtattgctgacaaaagcatagcggactgtgtggaagccctgctgggctgctatttaaccagctgtggggagagggctgctcagcttttcctctgttcactggggctgaaggtgctcccggtaattaaaaggactgatcgggaaaaggccctgtgccctactcgggagaatttcaacagccaacaaaagaacctttcagtgagctgtgctgctgcttctgtggccagttcacgctcttctgtattgaaagactcggaatatggttgtttgaagattccaccaagatgtatgtttgatcatccagatgcagataaaacactgaatcaccttatatcggggtttgaaaattttgaaaagaaaatcaactacagattcaagaataaggcttaccttctccaggcttttacacatgcctcctaccactacaatactatcactgattgttaccagcgcttagaattcctgggagatgcgattttggactacctcataaccaagcacctttatgaagacccgcggcagcactccccgggggtcctgacagacctgcggtctgccctggtcaacaacaccatctttgcatcgctggctgtaaagtacgactaccacaagtacttcaaagctgtctctcctgagctcttccatgtcattgatgactttgtgcagtttcagcttgagaagaatgaaatgcaaggaatggattctgagcttaggagatctgaggaggatgaagagaaagaagaggatattgaagttccaaaggccatgggggatatttttgagtcgcttgctggtgccatttacatggatagtgggatgtcactggagacagtctggcaggtgtactatcccatgatgcggccactaatagaaaagttttctgcaaatgtaccccgttcccctgtgcgagaattgcttgaaatggaaccagaaactgccaaatttagcccggctgagagaacttacgacgggaaggtcagagtcactgtggaagtagtaggaaaggggaaatttaaaggtgttggtcgaagttacaggattgccaaatctgcagcagcaagaagagccctccgaagcctcaaagctaatcaacctcaggttcccaatagctaa

SEQ ID NO: 20 - DNA, 20 bp, Artificial
  Synthetic primer rsGFP-F to amplify a gene encoding rsGFP
gccacaacattgaagatgga

SEQ ID NO: 21 - DNA, 20 bp, Artificial
  Synthetic primer rsGFP-R to amplify a gene encoding rsGFP
gaaagggcagattgtgtgga

SEQ ID NO: 22 - DNA, 20 bp, Artificial
  Synthetic primer Neo-F to amplify a gene encoding Neo
atagcgttggctacccgtga

SEQ ID NO: 23 - DNA, 20 bp, Artificial
  Synthetic primer Neo-R to amplify a gene encoding Neo
gaaggcgatagaaggcgatg

SEQ ID NO: 24 - DNA, 42 bp, Artificial
  Synthetic primer dsl-1 to amplify a gene encoding luciferase
gggtaatacgactcactatagggagaatggaagacgccaaaa

SEQ ID NO: 25 - DNA, 42 bp, Artificial
  Synthetic primer dsl-2 to amplify a gene encoding luciferase
gggtaatacgactcactatagggagagaacgtgtacatcgac

SEQ ID NO: 26 - DNA, 34 bp, Artificial
  Synthetic primer E to amplify a gene encoding CspB
gagcggataacatatgagaggaaaggttaagtgg

SEQ ID NO: 27 - DNA, 37 bp, Artificial
  Synthetic primer F to amplify a gene encoding CspB
gagcggataaggatccttactcaactactttcacgtg